(19) United States
(12) Patent Application Publication
(10) Pub. No.: US 2021/0145797 A1
(43) Pub. Date: May 20, 2021

(54) COMPOSITIONS FOR TREATING SKIN

(71) Applicant: UNIVERSITY OF FLORIDA RESEARCH FOUNDATION, INCORPORATED, Gainesville, FL (US)

(72) Inventor: Sadasivan VIDYASAGAR, Gainesville, FL (US)

(21) Appl. No.: 17/045,013

(22) PCT Filed: Apr. 4, 2019
(86) PCT No.: PCT/US19/25857

§ 371 Date: Oct. 2, 2020

Related U.S. Application Data
(60) Provisional application No. 62/652,776, filed on Apr. 4, 2018.

(51) Int. Cl. A61K 31/405; A61K 31/198; A61K 9/00

(52) U.S. Cl. CPC A61K 31/405; A61K 9/0014; A61K 31/198

(57) ABSTRACT

The present disclosure provides compositions and methods for promoting stem cell and/or progenitor cell proliferation and/or differentiation. The provided compositions useful in treating and/or preventing wounds or burns, treating and/or preventing skin conditions (e.g., atopic dermatitis, psoriasis, bed sores, conditions related to the aging of skin, pruritus, eczema or herpes simplex), or treating and/or preventing fibrosis, in a subject in need thereof. The present disclosure also provides methods for treating and/or preventing wounds or burns, treating and/or preventing skin conditions, or treating and/or preventing fibrosis in a subject in need of such treatment by administering a composition.

### RELATED APPLICATIONS

This application claims priority of U.S. Provisional Application No. 62/652,776, Apr. 4, 2018, the entirety of which is incorporated herein by reference for all purposes.

### BACKGROUND OF THE INVENTION

Skin diseases and conditions present a significant clinical challenge for medical practitioners and can cause significant physical discomfort and emotional distress for patients afflicted with such diseases and conditions. Such diseases and conditions of the skin may be temporary or chronic conditions. Despite the high prevalence of skin conditions in the population and the adverse effects of such conditions on quality-of-life and the economic burden associated therewith, there are few effective treatments for many skin conditions. Effective treatments for diseases and conditions of the skin, whether temporary or chronic in duration, represent an unmet need.

### SUMMARY OF THE INVENTION

Described herein are methods for treating and/or preventing a skin condition in a subject in need thereof comprising administering to the subject a composition comprising, consisting essentially of, or consisting of a therapeutically effective amount of free amino acids valine and serine; and a therapeutically effective amount of one or more free amino acids comprising tyrosine, aspartic acid, threonine, tryptophan, lysine or isoleucine, or a combination thereof.

In another embodiment of the method, the composition comprises, consists essentially of, or consists of a therapeutically effective amount of free amino acids valine and serine; and a therapeutically effective amount of two or more free amino acids comprising tyrosine, aspartic acid, threonine, tryptophan, lysine or isoleucine, or a combination thereof.

In another embodiment of the method, the composition comprises, consists essentially of, or consists of a therapeutically effective amount of free amino acids valine and serine; and a therapeutically effective amount of three or more free amino acids comprising tyrosine, aspartic acid, threonine, tryptophan, lysine or isoleucine, or a combination thereof.

In another embodiment of the method, the composition comprises, consists essentially of, or consists of a therapeutically effective amount of free amino acids valine and serine; and a therapeutically effective amount of four or more free amino acids comprising tyrosine, aspartic acid, threonine, tryptophan, lysine or isoleucine, or a combination thereof.

In another embodiment of the method, the composition comprises, consists essentially of, or consists of a therapeutically effective amount of free amino acids valine and serine; and a therapeutically effective amount of five or more free amino acids comprising tyrosine, aspartic acid, threonine, tryptophan, lysine or isoleucine, or a combination thereof.

In another embodiment of the method, the composition comprises, consists essentially of, or consists of a therapeutically effective amount of free amino acids valine, serine, tyrosine, aspartic acid, threonine, tryptophan, lysine and isoleucine.

In another embodiment of the method, the composition comprises, consists essentially of, or consists of a therapeutically effective amount of free amino acids valine and serine; and a therapeutically effective amount of one or more free amino acids comprising tyrosine, aspartic acid, or threonine, or a combination thereof.

In another embodiment of the method, the composition comprises, consists essentially of, or consists of a therapeutically effective amount of free amino acids valine and serine; and a therapeutically effective amount of two or more free amino acids comprising tyrosine, aspartic acid, or threonine, or a combination thereof.

In another embodiment of the method, the composition comprises, consists essentially of, or consists of a therapeutically effective amount of free amino acids valine, serine, tyrosine, aspartic acid, and threonine, or a combination thereof.

In another embodiment of the method, the composition comprises, consists essentially of, or consists of a therapeutically effective amount of free amino acids valine, serine, tyrosine, aspartic acid, threonine, and tryptophan, or a combination thereof.

In another embodiment of the method, the composition comprises equal to or less than 0.1 mg/L asparagine, equal to or less than 0.1 mg/L alanine, and/or equal to or less than 0.1 mg/L methionine. More particularly, the composition does not comprise asparagine, alanine, and/or methionine.

In another embodiment of the method, the skin condition comprises atopic dermatitis, dermatitis, psoriasis, aging of skin, a condition related to the aging of skin, bed sores, pruritus, eczema, or herpes simplex. In a more particular embodiment, the skin condition is a cosmetic skin condition. In another particular embodiment, the skin condition is fibrosis. In another particular embodiment, the skin condition is a wound or a burn.

Also encompassed herein is the use of a composition described herein for the treatment of a skin condition. In a further aspect, the composition comprises equal to or less than 0.1 mg/L asparagine, equal to or less than 0.1 mg/L alanine, and/or equal to or less than 0.1 mg/L methionine. In a still further aspect, the composition does not comprise asparagine, alanine, and/or methionine. In an embodiment of the use, the skin condition comprises atopic dermatitis, dermatitis, psoriasis, aging of skin, a condition related to the aging of skin, bed sores, pruritus, eczema, or herpes simplex. In another embodiment, the skin condition is a cosmetic skin condition. In another particular embodiment, the skin condition is fibrosis. In another particular embodiment, the skin condition is a wound or a burn.

Also encompassed herein is the use of a composition described herein in the preparation of a pharmaceutical composition for treating a skin condition in a subject in need thereof. In a further aspect, the composition comprises equal to or less than 0.1 mg/L asparagine, equal to or less than 0.1 mg/L alanine, and/or equal to or less than 0.1 mg/L methionine. In a still further aspect, the composition does not comprise asparagine, alanine, and/or methionine. In an embodiment of the use, the skin condition comprises atopic dermatitis, dermatitis, psoriasis, aging of skin, a condition related to the aging of skin, bed sores, pruritus, eczema, or herpes simplex. In another embodiment, the skin condition is a cosmetic skin condition. In another particular embodiment, the skin condition is fibrosis. In another particular embodiment, the skin condition is a wound or a burn.

In another embodiment of the method, the composition comprises, consists essentially of, or consists of a therapeutically effective amount of free amino acids lysine and aspartic acid; and optionally, a therapeutically effective amount of free amino acids valine. In another embodiment of the method, the composition comprises, consists essentially of, or consists of a therapeutically effective amount of free amino acids lysine, aspartic acid, and valine; and a therapeutically effective amount of one or more free amino acids comprising isoleucine, phenylalanine, tyrosine, tryptophan, cysteine, and glutamate, or a combination thereof. Use of this composition for the treatment of a skin condition or in the preparation of a pharmaceutical composition for treating a skin condition in a subject in need thereof are also encompassed. In a particular embodiment of the method or use, the composition comprises equal to or less than 0.1 mg/L asparagine, equal to or less than 0.1 mg/L alanine, and/or equal to or less than 0.1 mg/L methionine. More particularly, the composition does not comprise asparagine, alanine, and/or methionine.

In further aspect of the method or use, each of the free amino acids is at a concentration of from about 0.1 to 2.0 grams/liter.

In another further aspect of the method or use, the composition does not include, or comprises only negligible amounts of electrolytes.

In yet another aspect of the method or use, the subject is a human.

In a still further aspect of the method or use, the composition is administered to the subject via transdermal, subcutaneous, or topical administration.

In another further aspect of the method or use, the composition is administered on a continuous daily dosing schedule.

In another further aspect of the method or use, the composition is sterile.

In one aspect, the compositions described herein comprise at least one additional active agent.

In certain embodiments, the compositions described herein are in a form of a single unit dose. In one aspect, the compositions described herein have a pH of about 2.0 to about 8.5. According to one embodiment, the compositions described herein are formulated for transdermal, subcutaneous or topical administration.

Also described herein are kits, wherein the kit comprises a composition described herein and instructions for administering to a subject or contacting a biological sample with the composition.

### Definitions

The terms “improving skin condition” or “treating a skin condition” include therapeutically treating a skin condition, and may involve at least one of the following benefits: thickening of skin, preventing loss of skin elasticity, and a reduction in lines or winkles.

The term “epidermis” or “epidermal,” as used herein, refers to the outermost layer of the skin.

The term “topical application,” as used herein, means to apply or spread the compositions described herein onto the surface of the epidermis tissue.

The term “dermatologically-acceptable,” as used herein, means that the compositions or components thereof so described are suitable for use in contact with mammalian epidermal tissue without undue toxicity, incompatibility, instability, allergic response, and the like.

The term “therapeutically effective amount,” as used herein, refers to an amount of a compound or composition sufficient to induce a positive benefit, preferably an improvement in the appearance and/or texture of the skin. In accordance with the present disclosure, a therapeutically effective amount is an amount of amino acids, either alone or in combination with other agents, that regulates and/or improves the skin physically and/or visually.

The term “amelioration” or any grammatical variation thereof (e.g., ameliorate, ameliorating, and amelioration etc.), as used herein, includes, but is not limited to, delaying the onset, or reducing the severity of a disease or condition. Amelioration, as used herein, does not require the complete absence of symptoms.

The terms “effective amount” or “significant amount” as used herein, refers to an amount that is capable of treating or ameliorating a disease or condition or otherwise capable of producing an intended therapeutic effect.

The term “health functional food” refers to a food prepared or processed into tablet, capsule, powder, granule, liquid, pill, or any other form using raw materials or ingredients with useful functions for the human body.

The term “functional” means a useful effect for human health, such as structural or functional regulation of nutrients, the immune system, inflammation, fluid balance, physiological action, or the like.

The term “carrier” refers to a diluent, adjuvant, excipient, or vehicle with which the compound is administered. Examples of suitable pharmaceutical carriers are described in “Remington's Pharmaceutical Sciences” by E. W. Martin.

The term “treatment” or any grammatical variation thereof (e.g., treat, treating, and treatment etc.), as used herein, includes but is not limited to, alleviating a symptom of a disease or condition; and/or reducing, suppressing, inhibiting, lessening, or affecting the progression, severity, and/or scope of a disease or condition.

The term “consisting essentially of,” as used herein, limits the scope of the ingredients and steps to the specified materials or steps and those that do not materially affect the basic and novel characteristic(s) of the present invention, i.e., compositions and methods for promoting at least one of skin integrity, function, texture, or appearance.

### BRIEF DESCRIPTION OF THE DRAWINGS

FIG. 1A-C shows the relative transepithelial electrical resistance (TEER) of fully differentiated primary human keratinocytes after 1 hour incubation with single amino acids (AA) or amino acid combinations (5AA, 6AA and 8AA). Snapwell culture inserts with keratinocytes 120 hours post differentiation and incubated with AA are maintained in Ussing chambers containing isotonic Ringer solution. TEER was recorded after 0, 30, 60 and 90 min, and relative TEER was calculated for the time points 30 (FIG. 1A), 60 (FIG. 1B) and 90 min (FIG. 1C) in comparison to 0 min (Mean±SEM; n=4).

FIG. 2 shows survival fraction (SF) of human dermal fibroblasts after 1 hour and 4 hours incubation with single amino acids (AA) or amino acid combinations (5AA and 8AA). Fibroblasts were plated on 6-well culture plates in fibroblast basal media at a concentration of 500 cells/well. After 24 hours, cells were incubated with AA, and then maintained in fibroblast basal media for 14 days. After 14 days, the number of colonies was analyzed, and the survival fraction was calculated as followed: SF=PA_{AA}/PAR (PA: Plating efficiency; AA: amino acids; R: Ringer) with PA=colonies/cells (Mean±SEM; n=3).

FIG. 3 shows a keratinocytes summary: skin cell Ussing chamber studies. The results are presented in % data. All the data bars come from (90 min resistance)/(0 min resistance)*100%. Basic buffer is the control buffer for all 20 AAs.

FIG. 4 shows a keratinocytes summary: skin cell Ussing chamber studies. The results are presented in % data. All the data bars come from (90 min current)/(0 min current)*100%. Basic buffer is the control buffer for all 20 AAs.

FIG. 5A-F shows histogram plots of current relative to control as influenced by amino acid compositions, wherein the amino acids have been ranked based upon their barrier tightening property and secretory properties. The effect of the indicated additives on current are also depicted. The indicated additives may be used in, e.g., gel based medium, lotion, cream, etc.

FIG. 6A-F shows histogram plots of resistance relative to control as influenced by amino acid compositions, wherein the amino acids have been ranked based upon their barrier tightening property and secretory properties. The effect of the indicated additives on resistance are also depicted. The indicated additives may be used in, e.g., gel based medium, lotion, cream, etc.

### DETAILED DESCRIPTION

Described herein are compositions of amino acids for treating skin disorders. In one aspect, described herein are compositions and methods for promoting cellular proliferation and/or development. In a certain embodiment, the cells are stem cells and/or the progenitor cells. As used herein, reference to “development” can include, for example, migration, maturation, and/or differentiation of the cells. The disclosure also provides compositions and methods for treating and/or preventing a skin condition. Such skin conditions include, without limitation, atopic dermatitis, psoriasis, bed sores, conditions related to aging of the skin, bed sores, pruritis, eczema, herpes simplex, and/or a cosmetic condition. The disclosure also provides compositions and methods for treating and/or preventing fibrosis. The disclosure also provides compositions and methods for treating a wound or burn.

Atopic Dermatitis (AD), for example, is the most common chronic inflammatory skin disease in people, affecting up to 15 million Americans (17% of children and 6% adults). Despite its high prevalence, effects on quality-of-life, and economic burden, there are still few effective treatments for AD and most have focused generally on inhibiting inflammation. AD is thought to develop in part as consequence of an acquired or genetic defect of the skin's barrier. Indeed, the present inventors discovered that the epidermis from AD subjects exhibit altered tight junctions (TJ), which was associated with reduced expression of selected TJ components (e.g claudin). TJs seal the intercellular spaces between epithelial cells and the ‘tightness’ of this structure is dynamically regulated by endogenous or environmental factors. The regulation of the TJ seal is important for a variety of reasons, including appropriate trafficking patterns of ions, proteins, hydration, and even the penetration of immune cells.

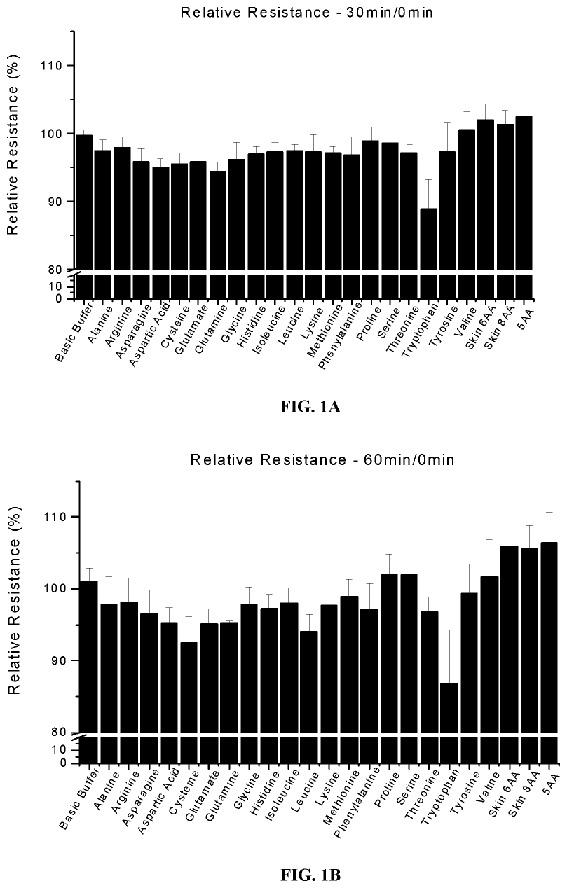

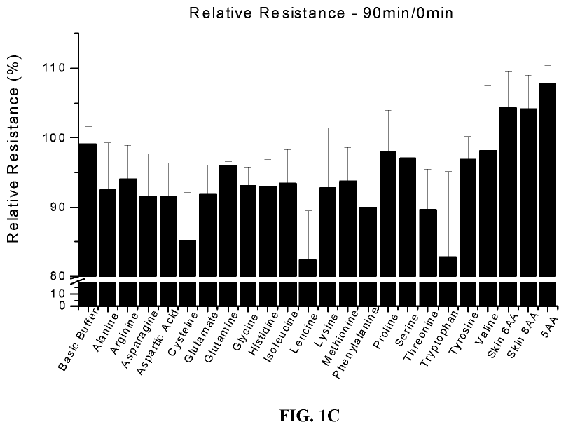

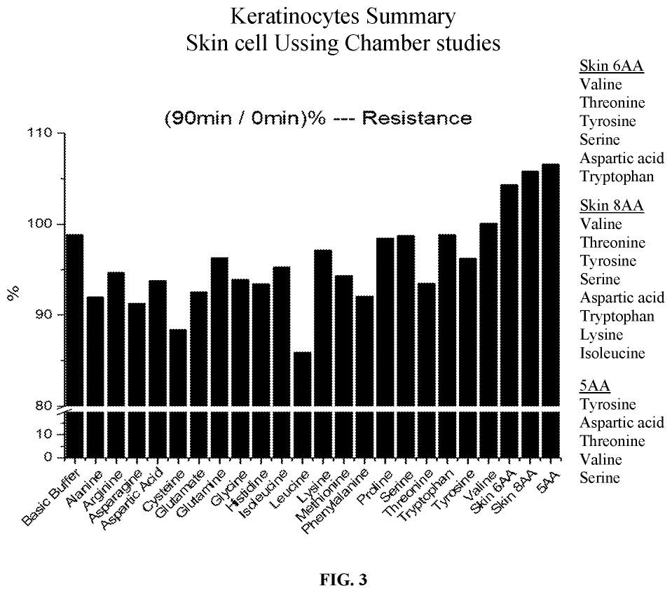

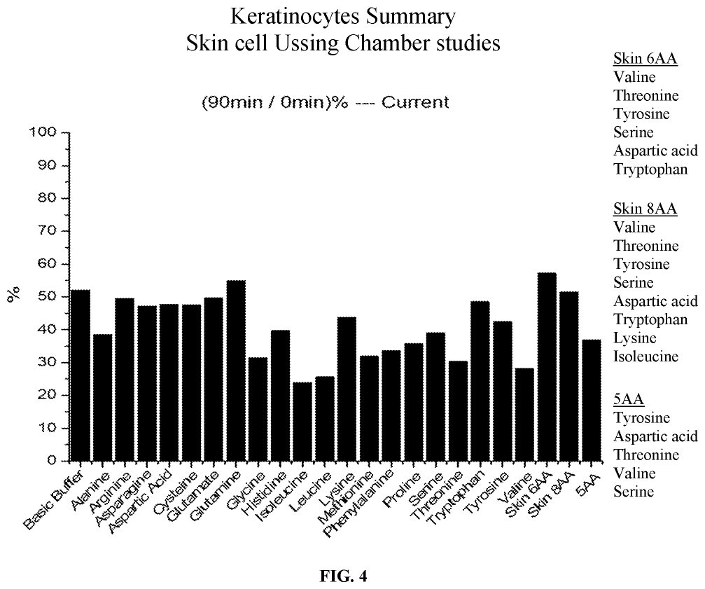

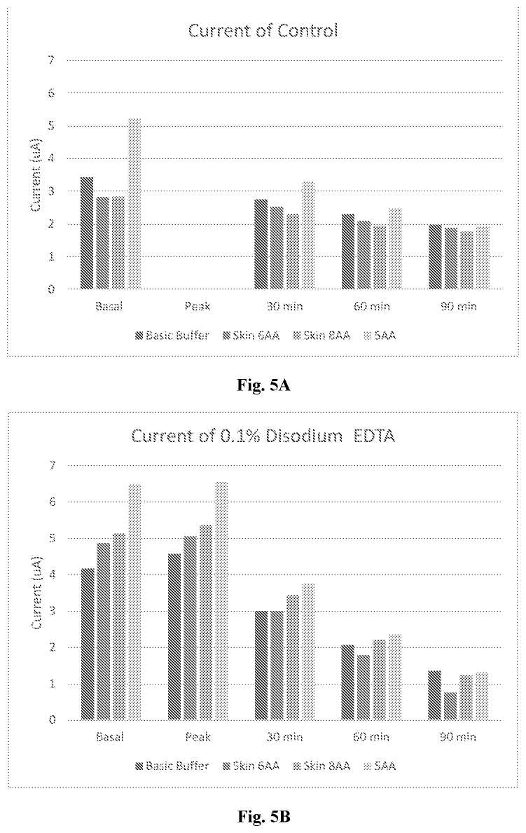

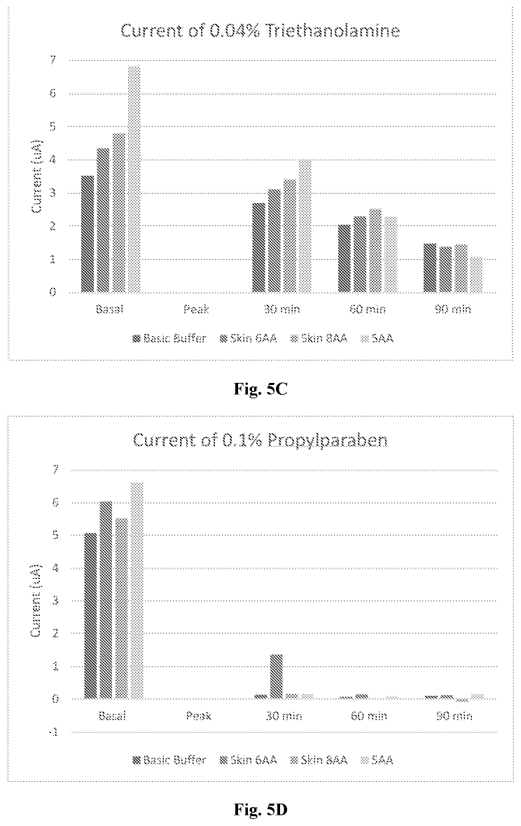

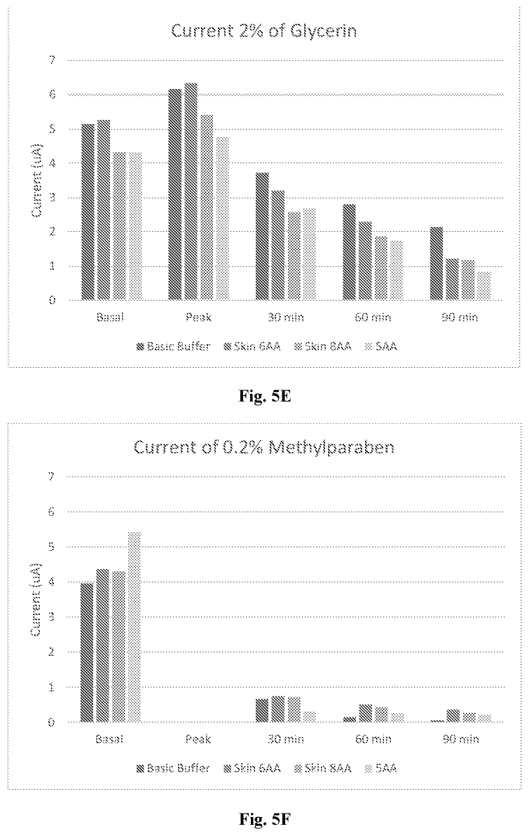

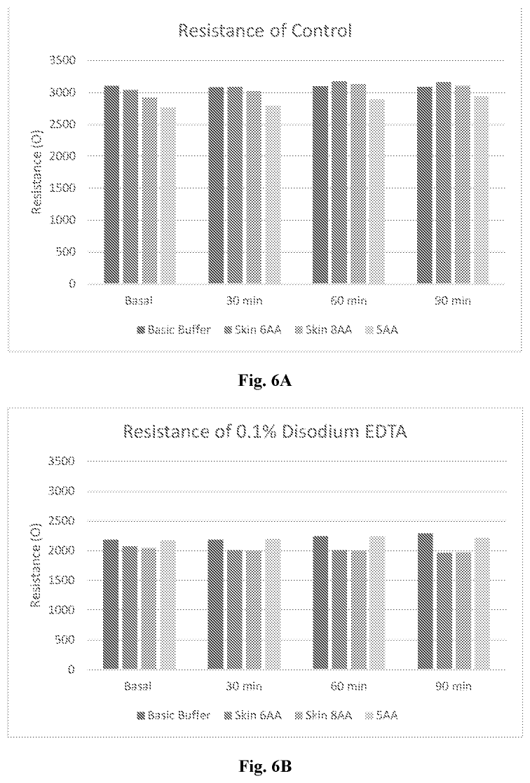

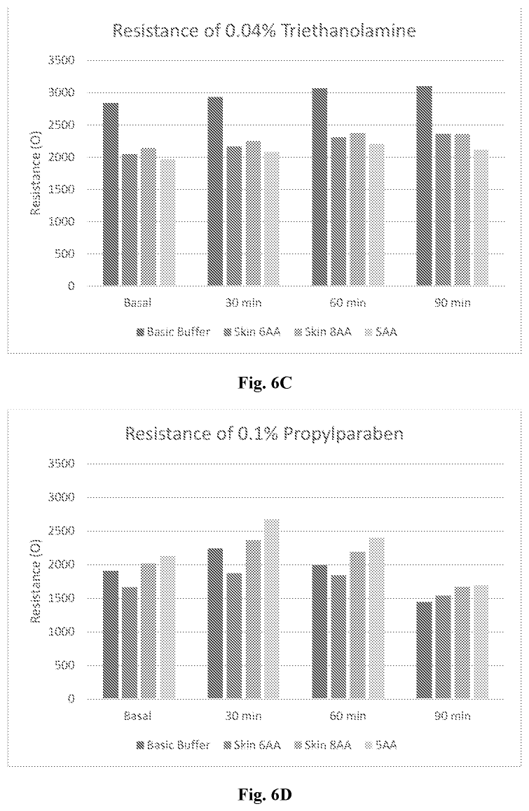

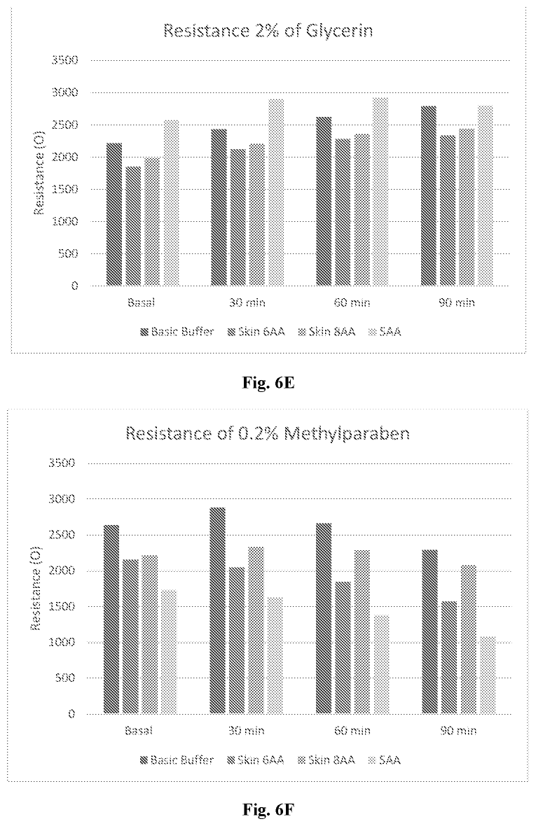

The integrity of the tight junction dynamics between endothelial cells and epithelial cells, which regulates diffusion and maintains homeostasis of organs protected by physiological barriers, may be measured in-vitro using transepithelial/transendothelial electrical resistance (TEER). TEER can be used to identify agents that improve physiological barriers, and that therefore may have a positive impact on conditions associated with elevated levels of permeability of physiological barriers relative to a normal, healthy state. Conditions associated with elevated levels of permeability of physiological barriers include, e.g., atopic dermatitis/xerosis and herpes simplex. Results presented herein reveal that certain amino acids and combinations thereof enhance integrity of the tight junction dynamics, thereby reducing permeability of tight junctions. See, for example, FIGS. 1-5.

Another mechanism to promote the process of tissue repair following insult to the or a disease of the skin is to enhance fibroblast proliferation. Identification of compositions that promote fibroblast proliferation may lead to identification of compositions that are useful in such indications as wound healing or combating the effects of aging. Conversely, identification of compositions that reduce fibroblast proliferation may lead to the identification of compositions useful in processes where fibroblast proliferation plays a role, such as scar formation following injury.

The subject may be a patient in which promoting the proliferation and/or development of stem cells and/or progenitor cells is needed. The patient may have this need due to, for example, physical trauma, malabsorption, inflammation, or infection. In one embodiment, the patient is asymptomatic. The subject can be any animal, including, for example, a human. In addition to humans, the animal may be, for example, mammals, such as rabbits, cattle, horses, sheep, pigs, goats, dogs, and cats. The animals may also be, for example, chickens, turkeys, or fish.

Stem Cells and/or Progenitor Cells

The compositions and methods can be used to enhance stem and/or progenitor cell populations in in vivo, ex vivo and/or in vitro. These cells are useful for providing treatment for many disease states, degeneration and injuries.

In one embodiment, provided herein are methods for promoting the proliferation and/or development of stem cells and/or the progenitor cells in a subject in need of such treatment by administering a composition of the present disclosure to the subject.

The compositions and methods described herein can be used to increase survival, proliferation, and/or development of stem cells and/or progenitor cells. The cells can be, for example, embryonic, pluripotent or totipotent, and can be in vivo or in vitro.

A stem cell is typically capable of differentiation into ectodermal, mesodermal, and endodermal cells. Pluripotent stem cells are undifferentiated cells that have the capability of differentiating into a variety of cell types. Totipotent stem cells are undifferentiated cells with the capability of differentiating into all cell types and, by definition, imply germline transmission.

In one embodiment, the stem cells are mesenchymal stem cells that have a potential to differentiate into, for example, osteoblasts, chondrocytes, adipocytes, fibroblasts, smooth muscle cells, stromal cells, tendon cells, epithelial cells, nerve cells, and vascular endothelial cells.

In one embodiment, the cells are embryonic stem (ES) cells, which can proliferate indefinitely in an undifferentiated state. Furthermore, ES cells are totipotent cells, meaning that they can generate all of the cells present in the body (bone, muscle, brain cells, etc.). ES cells have been isolated from the inner cell mass (ICM) of the developing murine blastocyst. Additionally, human cells with ES properties have been isolated from the inner blastocyst cell mass and developing germ cells. Human and non-human primate embryonic stem cells have been produced (see U.S. Pat. No. 6,200,806, which is incorporated by reference herein).

In one embodiment, the cells are adult stem cells, which self-renew and generate differentiated cells. Adult stem cells, also called somatic stem cells, are stem cells that maintain and repair the tissue in which they are found. These cells can be, for example, bone marrow stem cells.

Somatic precursor cells can also be utilized with the methods disclosed herein. Somatic precursor cells can be isolated from a variety of sources using methods known to one skilled in the art. Somatic precursor cells can be of ectodermal, mesodermal, or endodermal origin. Any somatic precursor cells that can be obtained and maintained in vitro can be used in accordance with the present methods. Such cells include cells of epithelial tissues such as the skin and the lining of the gut, embryonic heart muscle cells, and neural precursor cells. Such cells also include pancreatic stem cells, cord blood stem cells, peripheral blood stem cells, and stem cells derived from adipose tissues.

In one embodiment, the stem cells further include pluripotential stem cells obtained by reprogramming somatic cells. Somatic cell reprogramming is the process of converting the epigenetic state of a differentiated somatic cell into a pluripotent state capable of giving rise to any cell type. Somatic cell reprogramming can be achieved by for example, transferring a somatic nucleus into a donor oocyte, which is termed somatic cell nuclear transfer (SCNT). Somatic cell reprogramming can also be achieved by direct reprogramming, termed induced pluripotent stem cells (iPSCs), for example, by the simultaneous retroviral expression of the four transcription factors Oct4, Sox2, Klf4, and C-myc. These iPSCs share all the key characteristics of ES cells.

In another embodiment, other post-embryonic stem cells can be obtained beginning from week 12 after gestation from fetal liver, perinatal umbilical cord blood (UCB), human bone marrow or G-CSF stimulated peripheral blood.

In certain embodiments, the stem cells are skin stem cells, neural stem cells (NSCs), hematopoietic stem cells (HSCs), mesenchymal stem cells (MSCs), tissue stem cells (e.g., muscle stem cells), mesodermal stem cells, organ stem cells (e.g., pancreatic stem cells and liver stem cells), or intestinal stem cells. In certain embodiments, the stem cells are adult stem cells, embryonic stem cells, cancer stem cells, neural stem cells (NSCs), skin stem cells, hematopoietic stem cells (HSCs), mesenchymal stem cells (MSCs), tissue stem cells (e.g., muscle stem cells), mesodermal stem cells, organ stem cells (e.g., pancreatic stem cells and liver stem cells), or intestinal stem cells.

In one embodiment, the stem cells are hair follicle stem cells. The hair follicle bulge area is an abundant, easily accessible source of actively growing, pluripotent adult stem cells. Nestin, a protein marker for neural stem cells, is also expressed in follicle stem cells as well as in their immediate differentiated progeny. The nestin-expressing hair follicle stem cells differentiate into, for example, neurons, glial cells, keratinocytes, and smooth muscle cells in vitro.

In one embodiment, the cells are neuronal stem cells. In non-limiting examples, the cells are neuronal precursor cells and/or glial precursor cells. Undifferentiated neural stem cells differentiate into neuroblasts and glioblasts, which give rise to neurons and glial cells.

Neural stem and progenitor cells can participate in aspects of normal development, including migration along well-established migratory pathways to disseminated CNS regions, differentiation into multiple developmentally- and regionally-appropriate cell types in response to microenvironmental cues, and non-disruptive, non-tumorigenic interspersion with host progenitors and their progeny.

Human NSCs are capable of expressing foreign transgenes in vivo in these disseminated locations. As such, these cells find use in the treatment of a variety of conditions, including traumatic injury to the spinal cord, brain, and peripheral nervous system; treatment of degenerative disorders including Alzheimer's disease, Huntington's disease, and Parkinson's disease; affective disorders including major depression; stroke; and the like.

In one embodiment, the stem cells are muscle stem cells. Muscle tissue in adult vertebrates regenerates from reserve myoblasts called satellite cells. Satellite cells are distributed throughout muscle tissue and are mitotically quiescent in the absence of injury or disease. Following recovery from damage due to injury or disease or in response to stimuli for growth or hypertrophy, satellite cells reenter the cell cycle, proliferate and undergo differentiation into multinucleate myotubes, which form new muscle fiber. The myoblasts ultimately yield replacement muscle fibers or fuse into existing muscle fibers, thereby increasing fiber girth by the synthesis of contractile apparatus components. Criteria for myogenicity include the expression of myogenic proteins, which include the intermediate filament protein desmin, and myogenic transcription factors MyoD, Myf-5, and Pax-7.

In one embodiment, the stem cells are pancreatic stem cells and pancreatic multipotent progenitor (PMP) cells. These cells may be isolated from the pancreatic islet- and duct-derived tissue and further develop to, for example, other PMP cells or neural or pancreatic cells. The pancreatic cells optionally include alpha cells, delta cells, beta cells, pancreatic exocrine cells, and pancreatic stellate cells. α cells are mature glucagon producing cells. In vivo, these cells are found in the pancreatic islets of Langerhans. β cells are mature insulin producing cells. In vivo, these cells are also found in the pancreatic islets of Langerhans. Pancreatic stem cells are important in treatment of diabetes, in particular, type I diabetes, for providing β-cells.

In one embodiment, the stem cells are bone marrow stem cells. Bone marrow stem cells are cells that are generated in bone marrow and which can differentiate into cells of various body tissues. Bone marrow stem cells are also capable of recovering a lost function of a tissue by differentiating into cells of the tissue under the influence of a differentiation inducer. Examples of the bone marrow stem cells include bone marrow mesenchymal stem cells capable of differentiating into, for example, bone cells, chondrocytes, adipocytes, myocytes, tenocytes, or bone marrow stromal cells, and hematopoietic stem cells capable of differentiating into blood cells, such as erythrocytes and leukocytes.

### Compositions and Methods for Treating Skin Conditions

In certain embodiments, the present disclosure provides methods for treating and/or preventing a skin condition in a subject in need thereof, the method comprising administering to the subject a composition described herein. In certain aspects, the skin condition is atopic dermatitis, dermatitis, psoriasis, aging of skin, a condition related to the aging of skin (e.g., wrinkles, loss of suppleness, increase is roughness), bed sores, pruritus, eczema or herpes simplex. In further aspects, the skin condition is a cosmetic skin condition.

Herpes simplex is caused by the HSV-1 virus and periodically displays as a series of small blisters around the face or mouth, also called “cold sores” or “fever blisters”. This virus resides in the nervous system and becomes active during periods of stress or immune suppression, causing the blisters to form that break open and form small ulcers around the mouth and lips that take up to two to four seeks to heal and resolve. The blisters are accompanied by significant pain, burning, tenderness, and itching. The virus is easily transmitted during physical contact during this active phase of the condition. Genital herpes is similar and caused by the HSV-2 virus and occurs on genitalia. Anti-viral drugs (such as acyclovir) are used to treat these outbreaks, but only treat the symptoms of pain and itching, diminish the number of new outbreaks, and decrease the length of the time needed for the outbreak to resolve if administered in a timely fashion. The only over-the-counter topical treatment for herpes simplex is treatment with N-docosanol which speeds the time of healing from a median of 4.1 days to 2.5 days, an improvement of 1.5 days.

In certain aspects, the composition for treating and/or preventing a skin condition comprises, consists essentially of, or consists of, two or more free amino acids selected from the group consisting of valine, serine, threonine, tyrosine, aspartic acid. In a particular embodiment, the composition for treating and/or preventing a skin condition comprises, consists essentially of, or consists of free amino acids valine and serine and one or more free amino acids comprising tyrosine, aspartic acid, threonine, tryptophan, lysine and isoleucine. In a more particular embodiment, the composition for treating and/or preventing a skin condition comprises, consists essentially of, or consists of free amino acids valine and serine and two or more free amino acids comprising tyrosine, aspartic acid, threonine, tryptophan, lysine and isoleucine. In a more particular embodiment, the composition for treating and/or preventing a skin condition comprises, consists essentially of, or consists of free amino acids valine and serine and three or more free amino acids comprising tyrosine, aspartic acid, threonine, tryptophan, lysine and isoleucine. In a still more particular embodiment, the composition for treating and/or preventing a skin condition comprises, consists essentially of, or consists of free amino acids valine and serine and four or more free amino acids comprising tyrosine, aspartic acid, threonine, tryptophan, lysine and isoleucine. In a still more particular embodiment, the composition for treating and/or preventing a skin condition comprises, consists essentially of, or consists of free amino acids valine and serine and five or more free amino acids comprising tyrosine, aspartic acid, threonine, tryptophan, lysine and isoleucine. In a still more particular embodiment, the composition for treating and/or preventing a skin condition comprises, consists essentially of, or consists of free amino acids valine and serine and six free amino acids comprising tyrosine, aspartic acid, threonine, tryptophan, lysine and isoleucine.

In a more particular embodiment, the composition for treating and/or preventing a skin condition comprises, consists essentially of, or consists of free amino acids valine and serine and one or more free amino acids comprising tyrosine, aspartic acid, and threonine. In a more particular embodiment, the composition for treating and/or preventing a skin condition comprises, consists essentially of, or consists of free amino acids valine and serine and two or more free amino acids comprising tyrosine, aspartic acid, and threonine. In a still more particular embodiment, the composition for treating and/or preventing a skin condition comprises, consists essentially of, or consists of free amino acids valine and serine and three free amino acids comprising tyrosine, aspartic acid, and threonine.

In certain aspects, the composition for treating and/or preventing a skin condition comprises, consists essentially of, or consists of, one or more free amino acids selected from the group consisting of threonine, valine, tyrosine, tryptophan, aspartic acid, serine, lysine and isoleucine. In certain embodiments, the composition comprises one or more free amino acids selected from the group consisting of threonine, valine, tyrosine, tryptophan, aspartic acid, serine, lysine, isoleucine, and derivatives thereof. In certain embodiments, the composition comprises two or more free amino acids selected from the group consisting of threonine, valine, tyrosine, tryptophan, aspartic acid, serine, lysine and isoleucine. In certain embodiments, the composition comprises three or more free amino acids selected from the group consisting of threonine, valine, tyrosine, tryptophan, aspartic acid, serine, lysine and isoleucine. In certain embodiments, the composition comprises four or more free amino acids selected from the group consisting of threonine, valine, tyrosine, tryptophan, aspartic acid, serine, lysine and isoleucine. In certain embodiments, the composition comprises five or more free amino acids selected from the group consisting of threonine, valine, tyrosine, tryptophan, aspartic acid, serine, lysine and isoleucine. In certain embodiments, the composition comprises six or more free amino acids selected from the group consisting of threonine, valine, tyrosine, tryptophan, aspartic acid, serine, lysine and isoleucine. In certain embodiments, the composition comprises seven or more free amino acids selected from the group consisting of threonine, valine, tyrosine, tryptophan, aspartic acid, serine, lysine and isoleucine. In certain embodiments, the composition comprises threonine, valine, tyrosine, tryptophan, aspartic acid, serine, lysine and isoleucine. In certain embodiments, the composition comprises the free amino acids of threonine, valine, tyrosine, tryptophan, aspartic acid, serine, lysine and isoleucine.

In one embodiment, the composition comprises valine, threonine, tyrosine, serine and aspartic acid. In one embodiment, the composition comprises the free amino acids of valine, threonine, tyrosine, serine and aspartic acid.

In another embodiment, the composition comprises valine, threonine, tyrosine, serine, aspartic acid and tryptophan. In another embodiment, the composition comprises the free amino acids of valine, threonine, tyrosine, serine, aspartic acid and tryptophan.

In one embodiment, the composition comprises, consists essentially of, or consists of, one or more free amino acids selected from the group consisting of, valine, threonine, tyrosine, serine, aspartic acid, tryptophan, lysine and isoleucine, and derivatives thereof and optionally, for example, pharmaceutically acceptable carriers, adjuvants, and other active agents. In certain embodiments, the composition comprises, one or more free amino acids selected from the group consisting of, valine, threonine, tyrosine, serine, aspartic acid, tryptophan, lysine and isoleucine; and optionally, for example, pharmaceutically acceptable carriers, adjuvants, other active agents, and additives (e.g., sugars, electrolytes, vitamins, minerals, etc.).

In certain embodiments, the composition comprises one or more free amino acids selected from the group consisting of valine, tryptophan, serine, proline, lysine, glutamine, and tyrosine. In certain embodiments, the composition comprises one or more free amino acids selected from the group consisting of valine, tryptophan, serine, proline, lysine, glutamine, tyrosine, and derivatives thereof. In certain embodiments, the composition comprises two or more free amino acids selected from the group consisting of valine, tryptophan, serine, proline, lysine, glutamine, and tyrosine. In certain embodiments, the composition comprises three or more free amino acids selected from the group consisting of valine, tryptophan, serine, proline, lysine, glutamine, and tyrosine. In certain embodiments, the composition comprises four or more free amino acids selected from the group consisting of valine, tryptophan, serine, proline, lysine, glutamine, and tyrosine. In certain embodiments, the composition comprises five or more free amino acids selected from the group consisting of valine, tryptophan, serine, proline, lysine, glutamine, and tyrosine. In certain embodiments, the composition comprises six or more free amino acids selected from the group consisting of valine, tryptophan, serine, proline, lysine, glutamine, and tyrosine. In certain embodiments, the composition comprises valine, tryptophan, serine, proline, lysine, glutamine, and tyrosine. In certain embodiments, the composition comprises the free amino acids of valine, tryptophan, serine, proline, lysine, glutamine, and tyrosine.

In one embodiment, the therapeutic composition comprises, consists essentially of, or consists of, one or more free amino acids selected from the group consisting of, valine, tryptophan, serine, proline, lysine, glutamine, and tyrosine, and derivatives thereof; and optionally, for example, pharmaceutically acceptable carriers, adjuvants, and other active agents. In certain embodiments, the composition comprises, one or more free amino acids selected from the group consisting of, valine, threonine, tyrosine, serine, aspartic acid, tryptophan, lysine and isoleucine; and optionally, for example, pharmaceutically acceptable carriers, adjuvants, other active agents, and additives (e.g., sugars, electrolytes, vitamins, minerals, etc.).

In some specific embodiments, the composition does not include one or more amino acids selected from the group consisting of leucine, cysteine, asparagine, phenylalanine, alanine, glutamate, and histidine. In certain embodiments, the composition does not include leucine. In certain embodiments, the composition does not include cysteine. In certain embodiments, the composition does not include asparagine. In certain embodiments, the composition does not include phenylalanine. In certain embodiments, the composition does not include alanine. In certain embodiments, the composition does not include glutamate. In certain embodiments, the composition does not include histidine. In another specific embodiment, the composition does not include, or only includes negligible amounts of, leucine, cysteine, asparagine, phenylalanine, alanine, glutamate, and histidine.

Or, in certain embodiments, even if these amino acids are present in the composition, they are not present in an amount that would inhibit stem cell and/or progenitor cell survival, proliferation, and/or development. In some embodiments the composition has no serine, or negligible amounts of serine. By “negligible” it is meant that the specific amino acid present has no effect on stem cell survival, proliferation, and/or development. By “negligible” it is meant that the specific amino acid present has no effect on a disease or conditions that is related to mucosal barrier function, e.g., wound healing, treating skin conditions (e.g., atopic dermatitis, psoriasis, bed sores, or condition related to the aging of skin) in a subject in need thereof.

These amino acids, if present in the composition, may be present in, for example, the following concentrations: threonine at about 0.4 to about 1.5, about 0.7 to about 1.3, or about 0.9 to about 1.1 grams/liter; valine at about 0.7 to about 1.7, about 0.9 to about 1.5, or about 1.1 to about 1.3 grams/liter; serine at about 0.6 to about 1.6, about 0.8 to about 1.4, about 1.0 to about 1.2 grams/liter; tyrosine at about 0.05 to about 0.4, or about 0.1 to about 0.3 grams/liter; tryptophan at about 1.1 to about 2.1, about 1.3 to about 1.9, or about 1.5 to about 1.7 grams/liter; lysine at about 0.2 to about 1.2 grams/liter; and isoleucine at about 0.7 to about 1.7 grams/liter. In a certain embodiment, the composition comprises threonine (about 1.0 grams/liter), valine (about 1.2 grams/liter), serine (about 1.1 grams/liter), tyrosine (about 0.2 grams/liter), tryptophan (about 1.6 grams/liter), and aspartic acid (about 0.4 to 3.6 grams/liter). In certain embodiments, the composition has no, or negligible, serine. In certain embodiments, the concentration is grams amino acid per liter of solution. In certain embodiments, the solution comprises water.

These amino acids, if present in the composition, may be present in, for example, at a concentration of about 1 mM, about 2 mM, about 3 mM, about 4 mM, about 5 mM, about 6 mM, about 7 mM, about 8 mM, about 9 mM or about 10 mM. In preferred embodiments, the amino acids are at a concentration of about 8 mM. In one embodiment, glycine is at a concentration of about 8 mM, alanine is at a concentration of about 8 mM, leucine is at a concentration of about 8 mM, isoleucine is at a concentration of about 8 mM, proline is at a concentration of about 8 mM, phenylalanine is at a concentration of about 8 mM, tryptophan is at a concentration of about 8 mM, threonine is at a concentration of about 8 mM, cysteine is at a concentration of about 8 mM, methionine is at a concentration of about 8 mM, asparagine is at a concentration of about 8 mM, glutamine is at a concentration of about 8 mM, arginine is at a concentration of about 8 mM, histidine is at a concentration of about 8 mM, aspartate is at a concentration of about 8 mM, glutamate is at a concentration of about 8 mM, serine is at a concentration of about 10 mM, valine is at a concentration of about 10 mM, lysine is at a concentration of about 4 mM and tyrosine is at a concentration of 1.2 mM.

In one embodiment, the total osmolarity of the composition is from about 100 mosm to about 280 mosm, or preferably, about 150 to about 260 mosm.

The composition may have a pH ranging from about 2.5 to about 8.5. In certain embodiments, the pH of the composition ranges from about 2.5 to about 6.5, about 3.0 to about 6.0, about 3.5 to about 5.5, about 3.9 to about 5.0, or about 4.2 to about 4.6. In other embodiments, the pH of the composition ranges from about 6.5 to about 8.5, about 7.0 to about 8.0, or about 7.2 to about 7.8.

In certain embodiments, the composition has a pH from, for example, about 2.5 to about 8.5. In certain embodiments, the composition has a pH from about 2.5 to about 6.5, about 2.5 to about 6.0, about 3.0 to about 6.0, about 3.5 to about 6.0, about 3.9 to about 6.0, about 4.2 to about 6.0, about 3.5 to about 5.5, about 3.9 to about 5.0, or about 4.2 to about 4.6. In other embodiments, the pH is about 6.5 to about 8.5, about 7.0 to about 8.5, about 7.0 to about 8.0, about 7.2 to about 8.0, or about 7.2 to about 7.8.

In some embodiments, the composition is administered systemically or locally. In certain embodiments, the composition is used to promote cellular survival, proliferation, and/or development ex vivo or in vitro. In certain embodiments, the composition is used for treating a disease or conditions that is related to mucosal barrier function, e.g., wound healing, treating skin conditions (e.g., is atopic dermatitis, dermatitis, psoriasis, aging of skin, a condition related to the aging of skin, bed sores, pruritus, eczema or herpes simplex), and/or improving mucosal barrier function. The therapeutic composition can be administered via an enteral route or parenterally or topically or by inhalation. In certain embodiments, the composition is therapeutic, cosmetic, or nutritional.

In some embodiments, the composition is a solution. The composition can be administered with other therapeutic agents. In an embodiment, the compositions does not include glucose or includes negligible amounts of glucose. In one embodiment, the composition of the present disclosure does not include significant amounts of glucose, glutamine, methionine, and/or lactose. In certain embodiments, the composition does not include significant amounts of glucose. In certain embodiments, the composition does not include significant amounts of glutamine. In certain embodiments, the composition does not include significant amounts of methionine. In certain embodiments, the composition does not include methionine. In certain embodiments, the composition does not include significant amounts of lactose.

In one embodiment, the composition described herein is used as a composition for culturing cells for promoting survival, development, and/or proliferation of stem cells and/or progenitor cells. The composition for culturing cells may be used to obtain stem cells and/or progenitor cells in increased quantity in order to treat various diseases. The composition may also be applied to stem cells and/or progenitor cells immediately before and/or after transplantation. The composition may also be used to increase the proliferation of native stem cells present in various parts of the body.

In one embodiment, the present disclosure provides a method of improving therapeutic outcomes of implanted stem cells and/or progenitor cells comprising administering a composition in conjunction with stem cell and/or progenitor cell implantation or as a maintenance or supportive therapy following, for example, bone marrow or liver transplant. In certain embodiments, provided herein is a maintenance or supportive therapy following, for example, bone marrow or liver transplant. The administration of the composition can be at, or proximate to, a target stem and/or progenitor cell implantation site in a human or non-human animal. In an alternative embodiment, the environment may be further modified by providing influencing factors or by cleaning the environment of undesired or toxic agents that may affect administered stem and/or progenitor cells in an undesired way.

The administered cells may be unmodified or may be engineered to be biased toward a target differentiation endpoint. U.S. Patent Publication Nos. 2006/0134789 and 2006/0110440 are herein incorporated by reference in their entireties to provide examples of stem cells engineered for negative and positive differentiation biasing that are contemplated for use with the methods taught herein.

When the composition is applied to stem cells and/or progenitor cells either in culture, or in situ, changes occur in secretory proteins such as cell survival- and proliferation-related factors and transcription factors. As a result, the cellular activity is altered and, in particular, cell proliferation survival and/or development are enhanced. Accordingly, stem and/or progenitor cells produced in enhanced scale with the aid of the compositions described herein may be transplanted into a disease or other site as a cell therapy agent in order to promote regeneration of cells and effectively treat various conditions.

In one embodiment, the composition described herein stimulates the survival, proliferation, and/or development of stem cells and/or progenitor cells as evidenced by one or more of: 1) an increase in proliferation markers, such as p-ERK and p-AKT, at mRNA and/or protein levels, 2) an increase in stem cell markers, such as BMI1 and Lgr5, at mRNA and/or protein levels, 3) an activation of a protein kinase, such as MEK and ERK; and 4) a decrease in apoptosis markers, such as cleaved caspase 3.

ERK is a protein known to communicate cell surface signals to the nucleus for mediating the transcriptional and translational changes necessary to bring about proliferation. ERK1 and ERK2 are 44-kDa and 42-kDa proteins that are an important subfamily of protein kinases that control a broad range of cellular activities and physiological processes, including cell proliferation and differentiation by down-regulating pro-apoptotic molecules and upregulating anti-apoptotic molecules. Activation of MEK1/2 leads to the phosphorylation of ERK1 and ERK2. Upon stimulation, ERK1/2 becomes phosphorylated on threonine and tyrosine residues, and the latter results in the dissociation of ERK1/2 from MEK1/2. ERK1/2 then translocates to the nucleus. In one embodiment, the compositions described herein help maintain the mitogenic stimulus until late G1 for successful S-phase entry.

AKT is a serine/threonine-specific protein kinase that plays a role in cell proliferation and survival and inhibits apoptosis and metabolism. Phosphorylation of AKT activates AKT. Like pERK, AKT is also known to play a role in the cell cycle. AKT could also promote growth factor-mediated cell survival. The Akt pathway plays in important role in preventing apoptotic cell death. PCNA, a distinctive protein linked to DNA replication, is used as a marker for proliferation.

Compositions and Methods for Treating and/or Preventing Fibrosis

In one aspect, provided herein are methods for treating and/or preventing fibrosis, the method comprising administering to the subject a composition described herein. As used herein, fibrosis refers to the thickening and scarring of connective tissue, which may result from injury.

In certain aspects, the composition for treating and/or preventing fibrosis comprises, consists essentially of, or consists of, two or more free amino acids selected from the group consisting of valine, serine, threonine, tyrosine, aspartic acid. In a particular embodiment, the composition for treating and/or preventing fibrosis comprises, consists essentially of, or consists of free amino acids valine and serine and one or more free amino acids comprising tyrosine, aspartic acid, threonine, tryptophan, lysine and isoleucine. In a more particular embodiment, the composition for treating and/or preventing fibrosis comprises, consists essentially of, or consists of free amino acids valine and serine and two or more free amino acids comprising tyrosine, aspartic acid, threonine, tryptophan, lysine and isoleucine. In a more particular embodiment, the composition for treating and/or preventing fibrosis comprises, consists essentially of, or consists of free amino acids valine and serine and three or more free amino acids comprising tyrosine, aspartic acid, threonine, tryptophan, lysine and isoleucine. In a still more particular embodiment, the composition for treating and/or preventing fibrosis comprises, consists essentially of, or consists of free amino acids valine and serine and four or more free amino acids comprising tyrosine, aspartic acid, threonine, tryptophan, lysine and isoleucine. In a still more particular embodiment, the composition for treating and/or preventing fibrosis comprises, consists essentially of, or consists of free amino acids valine and serine and five or more free amino acids comprising tyrosine, aspartic acid, threonine, tryptophan, lysine and isoleucine. In a still more particular embodiment, the composition for treating and/or preventing fibrosis comprises, consists essentially of, or consists of free amino acids valine and serine and six free amino acids comprising tyrosine, aspartic acid, threonine, tryptophan, lysine and isoleucine.

In a more particular embodiment, the composition for treating and/or preventing fibrosis comprises, consists essentially of, or consists of free amino acids valine and serine and one or more free amino acids comprising tyrosine, aspartic acid, and threonine. In a more particular embodiment, the composition for treating and/or preventing fibrosis comprises, consists essentially of, or consists of free amino acids valine and serine and two or more free amino acids comprising tyrosine, aspartic acid, and threonine. In a still more particular embodiment, the composition for treating and/or preventing fibrosis comprises, consists essentially of, or consists of free amino acids valine and serine and three free amino acids comprising tyrosine, aspartic acid, and threonine.

In certain embodiments, the composition for treating and/or preventing fibrosis comprises one or more free amino acids selected from the group consisting of valine, threonine, tyrosine, serine, aspartic acid, lysine, isoleucine, glycine, and derivatives thereof. In certain embodiments, the composition comprises two or more free amino acids selected from the group consisting of valine, threonine, tyrosine, serine, aspartic acid, lysine, isoleucine, and glycine. In certain embodiments, the composition comprises three or more free amino acids selected from the group consisting of valine, threonine, tyrosine, serine, aspartic acid, lysine, isoleucine, and glycine. In certain embodiments, the composition comprises four or more free amino acids selected from the group consisting of valine, threonine, tyrosine, serine, aspartic acid, lysine, isoleucine, and glycine. In certain embodiments, the composition comprises five or more free amino acids selected from the group consisting of valine, threonine, tyrosine, serine, aspartic acid, lysine, isoleucine, and glycine. In certain embodiments, the composition comprises six or more free amino acids selected from the group consisting of valine, threonine, tyrosine, serine, aspartic acid, lysine, isoleucine, and glycine. In certain embodiments, the composition comprises seven or more free amino acids selected from the group consisting of valine, threonine, tyrosine, serine, aspartic acid, lysine, isoleucine, and glycine. In certain embodiments, the composition comprises valine, threonine, tyrosine, serine, aspartic acid, lysine, isoleucine, and glycine. In certain embodiments, the composition comprises the free amino acids of valine, threonine, tyrosine, serine, aspartic acid, lysine, isoleucine, and glycine. In one embodiment, the composition comprises valine, threonine, tyrosine, serine and aspartic acid. In certain embodiments, the composition comprises the free amino acids of valine, threonine, tyrosine, serine and aspartic acid.

In certain aspects, the composition for treating and/or preventing fibrosis further comprises methionine and/or asparagine.

In certain aspects, the composition for treating and/or preventing fibrosis comprises, consists essentially of, or consists of, one or more free amino acids selected from the group consisting of glycine, methionine, tyrosine, tryptophan, proline, phenylalanine and glutamate. In certain embodiments, the composition for treating and/or preventing fibrosis comprises one or more free amino acids selected from the group consisting of glycine, methionine, tyrosine, tryptophan, proline, phenylalanine and glutamate, and derivatives thereof. In certain embodiments, the composition comprises two or more free amino acids selected from the group consisting of glycine, methionine, tyrosine, tryptophan, proline, phenylalanine and glutamate. In certain embodiments, the composition comprises three or more free amino acids selected from the group consisting of glycine, methionine, tyrosine, tryptophan, proline, phenylalanine and glutamate. In certain embodiments, the composition comprises four or more free amino acids selected from the group consisting of glycine, methionine, tyrosine, tryptophan, proline, phenylalanine and glutamate. In certain embodiments, the composition comprises five or more free amino acids selected from the group consisting of glycine, methionine, tyrosine, tryptophan, proline, phenylalanine and glutamate. In certain embodiments, the composition comprises six or more free amino acids selected from the group consisting of glycine, methionine, tyrosine, tryptophan, proline, phenylalanine and glutamate. In certain embodiments, the composition comprises glycine, methionine, tyrosine, tryptophan, proline, phenylalanine and glutamate. In certain embodiments, the composition comprises the free amino acids of glycine, methionine, tyrosine, tryptophan, proline, phenylalanine and glutamate. In one embodiment, the composition comprises glycine, methionine, tyrosine, tryptophan, proline, phenylalanine and glutamate. In certain embodiments, the composition comprises the free amino acids of glycine, methionine, tyrosine, tryptophan, proline, phenylalanine and glutamate.

In certain aspects, the composition for treating and/or preventing fibrosis comprises, consists essentially of, or consists of, one or more free amino acids selected from the group consisting of glycine, threonine, glutamine, phenylalanine and proline. In certain embodiments, the composition for treating and/or preventing fibrosis comprises one or more free amino acids selected from the group consisting of glycine, threonine, glutamine, phenylalanine and proline, and derivatives thereof. In certain embodiments, the composition comprises two or more free amino acids selected from the group consisting of glycine, threonine, glutamine, phenylalanine and proline. In certain embodiments, the composition comprises three or more free amino acids selected from the group consisting of glycine, threonine, glutamine, phenylalanine and proline. In certain embodiments, the composition comprises four or more free amino acids selected from the group consisting of glycine, threonine, glutamine, phenylalanine and proline. In certain embodiments, the composition comprises glycine, threonine, glutamine, phenylalanine and proline. In certain embodiments, the composition comprises the free amino acids of glycine, threonine, glutamine, phenylalanine and proline.

These amino acids, if present in the composition, may be present in, for example, the following concentrations: threonine at about 0.4 to about 1.5, about 0.7 to about 1.3, or about 0.9 to about 1.1 grams/liter; valine at about 0.7 to about 1.7, about 0.9 to about 1.5, or about 1.1 to about 1.3 grams/liter; serine at about 0.6 to about 1.6, about 0.8 to about 1.4, about 1.0 to about 1.2 grams/liter; tyrosine at about 0.05 to about 0.4, or about 0.1 to about 0.3 grams/liter; and tryptophan at about 1.1 to about 2.1, about 1.3 to about 1.9, or about 1.5 to about 1.7 grams/liter. In a certain embodiment, the composition comprises threonine (about 1.0 grams/liter), valine (about 1.2 grams/liter), serine (about 1.1 grams/liter), tyrosine (about 0.2 grams/liter), tryptophan (about 1.6 grams/liter), and aspartic acid (about 0.4 to 3.6 grams/liter). In certain embodiments, the concentration is grams amino acid per liter of solution. In certain embodiments, the solution comprises water.

These amino acids, if present in the composition, may be present in, for example, at a concentration of about 1 mM, about 2 mM, about 3 mM, about 4 mM, about 5 mM, about 6 mM, about 7 mM, about 8 mM, about 9 mM or about 10 mM. In preferred embodiments, the amino acids are at a concentration of about 8 mM. In one embodiment, glycine is at a concentration of about 8 mM, alanine is at a concentration of about 8 mM, leucine is at a concentration of about 8 mM, isoleucine is at a concentration of about 8 mM, proline is at a concentration of about 8 mM, phenylalanine is at a concentration of about 8 mM, tryptophan is at a concentration of about 8 mM, threonine is at a concentration of about 8 mM, cysteine is at a concentration of about 8 mM, methionine is at a concentration of about 8 mM, asparagine is at a concentration of about 8 mM, glutamine is at a concentration of about 8 mM, arginine is at a concentration of about 8 mM, histidine is at a concentration of about 8 mM, aspartate is at a concentration of about 8 mM, glutamate is at a concentration of about 8 mM, serine is at a concentration of about 10 mM, valine is at a concentration of about 10 mM, lysine is at a concentration of about 4 mM and tyrosine is at a concentration of 1.2 mM.

In one embodiment, the total osmolarity of the composition is from about 100 mosm to about 280 mosm, or preferably, about 150 to about 260 mosm.

The composition may have a pH ranging from about 2.5 to about 8.5. In certain embodiments, the pH of the composition ranges from about 2.5 to about 6.5, about 3.0 to about 6.0, about 3.5 to about 5.5, about 3.9 to about 5.0, or about 4.2 to about 4.6. In other embodiments, the pH of the composition ranges from about 6.5 to about 8.5, about 7.0 to about 8.0, or about 7.2 to about 7.8.

In certain embodiments, the composition has a pH from, for example, about 2.5 to about 8.5. In certain embodiments, the composition has a pH from about 2.5 to about 6.5, about 2.5 to about 6.0, about 3.0 to about 6.0, about 3.5 to about 6.0, about 3.9 to about 6.0, about 4.2 to about 6.0, about 3.5 to about 5.5, about 3.9 to about 5.0, or about 4.2 to about 4.6. In other embodiments, the pH is about 6.5 to about 8.5, about 7.0 to about 8.5, about 7.0 to about 8.0, about 7.2 to about 8.0, or about 7.2 to about 7.8.

In some embodiments, the composition is administered systemically or locally. The therapeutic composition can be administered via an enteral route or parenterally or topically or by inhalation. In certain embodiments, the composition is therapeutic, cosmetic, or nutritional.

Compositions and Methods for Treating and/or Preventing a Wound, Burn, a Condition Relating to Aging of Skin or Herpes Simplex

In certain embodiments, the present disclosure provides methods for treating and/or preventing a wound, burn, a condition relating to aging of the skin or herpes simplex, the method comprising administering to the subject a composition described herein.

In certain aspects, the composition for treating and/or preventing a wound, burn, a condition relating to aging of the skin, or herpes simplex comprises, consists essentially of, or consists of, two or more free amino acids selected from the group consisting of valine, serine, threonine, tyrosine, aspartic acid. In a particular embodiment, the composition for treating and/or preventing a wound, burn, a condition relating to aging of the skin, or herpes simplex comprises, consists essentially of, or consists of free amino acids valine and serine and one or more free amino acids comprising tyrosine, aspartic acid, threonine, tryptophan, lysine and isoleucine. In a more particular embodiment, the composition for treating and/or preventing a wound, burn, a condition relating to aging of the skin, or herpes simplex comprises, consists essentially of, or consists of free amino acids valine and serine and two or more free amino acids comprising tyrosine, aspartic acid, threonine, tryptophan, lysine and isoleucine. In a more particular embodiment, the composition for treating and/or preventing a wound, burn, a condition relating to aging of the skin, or herpes simplex comprises, consists essentially of, or consists of free amino acids valine and serine and three or more free amino acids comprising tyrosine, aspartic acid, threonine, tryptophan, lysine and isoleucine. In a still more particular embodiment, the composition for treating and/or preventing a wound, burn, a condition relating to aging of the skin, or herpes simplex comprises, consists essentially of, or consists of free amino acids valine and serine and four or more free amino acids comprising tyrosine, aspartic acid, threonine, tryptophan, lysine and isoleucine. In a still more particular embodiment, the composition for treating and/or preventing a wound, burn, a condition relating to aging of the skin, or herpes simplex comprises, consists essentially of, or consists of free amino acids valine and serine and five or more free amino acids comprising tyrosine, aspartic acid, threonine, tryptophan, lysine and isoleucine. In a still more particular embodiment, the composition for treating and/or preventing a wound, burn, a condition relating to aging of the skin, or herpes simplex comprises, consists essentially of, or consists of free amino acids valine and serine and six free amino acids comprising tyrosine, aspartic acid, threonine, tryptophan, lysine and isoleucine.

In a more particular embodiment, the composition for treating and/or preventing a wound, burn, a condition relating to aging of the skin, or herpes simplex comprises, consists essentially of, or consists of free amino acids valine and serine and one or more free amino acids comprising tyrosine, aspartic acid, and threonine. In a more particular embodiment, the composition for treating and/or preventing a wound, burn, a condition relating to aging of the skin, or herpes simplex comprises, consists essentially of, or consists of free amino acids valine and serine and two or more free amino acids comprising tyrosine, aspartic acid, and threonine. In a still more particular embodiment, the composition for treating and/or preventing a wound, burn, a condition relating to aging of the skin, or herpes simplex comprises, consists essentially of, or consists of free amino acids valine and serine and three free amino acids comprising tyrosine, aspartic acid, and threonine.

In certain aspects, the composition for treating and/or preventing a wound, burn, a condition relating to aging of the skin, or herpes simplex comprises, consists essentially of, or consists of, one or more free amino acids selected from the group consisting of asparagine, valine, isoleucine, alanine and arginine.

In certain aspects, the composition for treating and/or preventing a wound, burn, or condition relating to aging of the skin comprises, consists essentially of, or consists of, one or more free amino acids selected from the group consisting of asparagine, valine, isoleucine, alanine and arginine or derivatives thereof. In certain embodiments, the composition comprises two or more free amino acids selected from the group consisting of asparagine, valine, isoleucine, alanine and arginine. In certain embodiments, the composition comprises three or more free amino acids selected from the group consisting of asparagine, valine, isoleucine, alanine and arginine. In certain embodiments, the composition comprises four or more free amino acids selected from the group consisting of asparagine, valine, isoleucine, alanine and arginine. In certain embodiments, the composition comprises asparagine, valine, isoleucine, alanine and arginine. In certain embodiments, the composition comprises the free amino acids of asparagine, valine, isoleucine, alanine and arginine.

In one embodiment, the therapeutic composition comprises, consists essentially of, or consists of, one or more free amino acids selected from the group consisting of, asparagine, valine, isoleucine, alanine and arginine, and derivatives thereof; and optionally, for example, pharmaceutically acceptable carriers, adjuvants, and other active agents. In certain embodiments, the composition comprises, one or more free amino acids selected from the group consisting of, asparagine, valine, isoleucine, alanine and arginine; and optionally, for example, pharmaceutically acceptable carriers, adjuvants, other active agents, and additives (e.g., sugars, electrolytes, vitamins, minerals, etc.).

These amino acids, if present in the composition, may be present in, for example, the following concentrations: threonine at about 0.4 to about 1.5, about 0.7 to about 1.3, or about 0.9 to about 1.1 grams/liter; valine at about 0.7 to about 1.7, about 0.9 to about 1.5, or about 1.1 to about 1.3 grams/liter; serine at about 0.6 to about 1.6, about 0.8 to about 1.4, about 1.0 to about 1.2 grams/liter; tyrosine at about 0.05 to about 0.4, or about 0.1 to about 0.3 grams/liter; and tryptophan at about 1.1 to about 2.1, about 1.3 to about 1.9, or about 1.5 to about 1.7 grams/liter. In a certain embodiment, the composition comprises threonine (about 1.0 grams/liter), valine (about 1.2 grams/liter), serine (about 1.1 grams/liter), tyrosine (about 0.2 grams/liter), tryptophan (about 1.6 grams/liter), and aspartic acid (about 0.4 to 3.6 grams/liter). In certain embodiments, the composition has no, or negligible, serine. In certain embodiments, the concentration is grams amino acid per liter of solution. In certain embodiments, the solution comprises water.

These amino acids, if present in the composition, may be present in, for example, at a concentration of about 1 mM, about 2 mM, about 3 mM, about 4 mM, about 5 mM, about 6 mM, about 7 mM, about 8 mM, about 9 mM or about 10 mM. In preferred embodiments, the amino acids are at a concentration of about 8 mM. In one embodiment, glycine is at a concentration of about 8 mM, alanine is at a concentration of about 8 mM, leucine is at a concentration of about 8 mM, isoleucine is at a concentration of about 8 mM, proline is at a concentration of about 8 mM, phenylalanine is at a concentration of about 8 mM, tryptophan is at a concentration of about 8 mM, threonine is at a concentration of about 8 mM, cysteine is at a concentration of about 8 mM, methionine is at a concentration of about 8 mM, asparagine is at a concentration of about 8 mM, glutamine is at a concentration of about 8 mM, arginine is at a concentration of about 8 mM, histidine is at a concentration of about 8 mM, aspartate is at a concentration of about 8 mM, glutamate is at a concentration of about 8 mM, serine is at a concentration of about 10 mM, valine is at a concentration of about 10 mM, lysine is at a concentration of about 4 mM and tyrosine is at a concentration of 1.2 mM.

In one embodiment, the total osmolarity of the composition is from about 100 mosm to about 280 mosm, or preferably, about 150 to about 260 mosm.

The composition may have a pH ranging from about 2.5 to about 8.5. In certain embodiments, the pH of the composition ranges from about 2.5 to about 6.5, about 3.0 to about 6.0, about 3.5 to about 5.5, about 3.9 to about 5.0, or about 4.2 to about 4.6. In other embodiments, the pH of the composition ranges from about 6.5 to about 8.5, about 7.0 to about 8.0, or about 7.2 to about 7.8.

In certain embodiments, the composition has a pH from, for example, about 2.5 to about 8.5. In certain embodiments, the composition has a pH from about 2.5 to about 6.5, about 2.5 to about 6.0, about 3.0 to about 6.0, about 3.5 to about 6.0, about 3.9 to about 6.0, about 4.2 to about 6.0, about 3.5 to about 5.5, about 3.9 to about 5.0, or about 4.2 to about 4.6. In other embodiments, the pH is about 6.5 to about 8.5, about 7.0 to about 8.5, about 7.0 to about 8.0, about 7.2 to about 8.0, or about 7.2 to about 7.8.

In some embodiments, the composition is administered systemically or locally. The therapeutic composition can be administered via an enteral route or parenterally or topically or by inhalation. In certain embodiments, the composition is therapeutic, cosmetic, or nutritional.

Compositions for Promoting Proliferation and/or Development of Stem Cells and/or Progenitor Cells, and for Wound Healing, Treating Skin Conditions, Mucosal Barrier Conditions, and a Disease or Conditions that are Related to Mucosal Barrier Function

In one aspect, provided herein are therapeutic compositions for promoting the survival, proliferation, and/or development of stem cells and/or progenitor cells. Described herein are compositions and methods for treating a disease or conditions that are related to mucosal barrier function, e.g., wound healing, treating skin conditions (e.g., atopic dermatitis, psoriasis, bed sores, or condition related to the aging of skin), and/or improving mucosal barrier function in a subject in need thereof.

In one embodiment, the therapeutic composition comprises, consists essentially of, or consists of, one or more free amino acids selected from the group consisting of, threonine, valine, tyrosine, tryptophan, aspartic acid, serine and derivatives thereof; and optionally, pharmaceutically acceptable carriers, adjuvants, and/or additional active ingredients. In one embodiment, the therapeutic composition comprises, consists essentially of, or consists of, one or more free amino acids selected from the group consisting of, threonine, valine, tyrosine, tryptophan, aspartic acid, and serine; and optionally, pharmaceutically acceptable carriers, adjuvants, and/or additional active ingredients. In one embodiment, the therapeutic composition comprises, consists essentially of, or consists of, two or more free amino acids selected from the group consisting of valine, serine, threonine, tyrosine, aspartic acid; and optionally, pharmaceutically acceptable carriers, adjuvants, and/or additional active ingredients. In one embodiment, the therapeutic composition comprises, consists essentially of, or consists of free amino acids valine and serine and one or more free amino acids comprising tyrosine, aspartic acid, threonine, tryptophan, lysine and isoleucine; and optionally, pharmaceutically acceptable carriers, adjuvants, and/or additional active ingredients. In one embodiment, the therapeutic composition comprises, consists essentially of, or consists of free amino acids valine and serine and two or more free amino acids comprising tyrosine, aspartic acid, threonine, tryptophan, lysine and isoleucine; and optionally, pharmaceutically acceptable carriers, adjuvants, and/or additional active ingredients. In one embodiment, the therapeutic composition comprises, consists essentially of, or consists of free amino acids valine and serine and three or more free amino acids comprising tyrosine, aspartic acid, threonine, tryptophan, lysine and isoleucine; and optionally, pharmaceutically acceptable carriers, adjuvants, and/or additional active ingredients. In one embodiment, the therapeutic composition comprises, consists essentially of, or consists of free amino acids valine and serine and four or more free amino acids comprising tyrosine, aspartic acid, threonine, tryptophan, lysine and isoleucine; and optionally, pharmaceutically acceptable carriers, adjuvants, and/or additional active ingredients. In one embodiment, the therapeutic composition comprises, consists essentially of, or consists of free amino acids valine and serine and five or more free amino acids comprising tyrosine, aspartic acid, threonine, tryptophan, lysine and isoleucine; and optionally, pharmaceutically acceptable carriers, adjuvants, and/or additional active ingredients. In one embodiment, the therapeutic composition comprises, consists essentially of, or consists of free amino acids valine and serine and six free amino acids comprising tyrosine, aspartic acid, threonine, tryptophan, lysine and isoleucine; and optionally, pharmaceutically acceptable carriers, adjuvants, and/or additional active ingredients. In certain embodiments, the composition is sterile.

In one embodiment, the therapeutic composition comprises, consists essentially of, or consists of free amino acids valine and serine and one or more free amino acids comprising tyrosine, aspartic acid, and threonine; and optionally, pharmaceutically acceptable carriers, adjuvants, and/or additional active ingredients. In one embodiment, the therapeutic composition comprises, consists essentially of, or consists of free amino acids valine and serine and two or more free amino acids comprising tyrosine, aspartic acid, and threonine; and optionally, pharmaceutically acceptable carriers, adjuvants, and/or additional active ingredients. In one embodiment, the therapeutic composition comprises, consists essentially of, or consists of free amino acids valine and serine and three free amino acids comprising tyrosine, aspartic acid, and threonine; and optionally, pharmaceutically acceptable carriers, adjuvants, and/or additional active ingredients. In certain embodiments, the composition is sterile.

In one embodiment, the total osmolarity of the composition is from about 100 mosm to about 280 mosm, or any value therebetween. Preferably, the total osmolarity is from about 150 to about 260 mosm. In another embodiment, the composition has a total osmolarity that is any value lower than about 280 mosm.

The composition may have a pH from, for example 2.5 to 8.5. In certain embodiments, the composition has a pH from about 2.5 to about 6.5, about 3.0 to about 6.0, about 3.5 to about 5.5, about 3.9 to about 5.0, or about 4.2 to about 4.6. In other embodiments, the pH is about 6.5 to about 8.5, about 7.0 to about 8.0, or about 7.2 to about 7.8.

In certain embodiments, the composition comprises one or more free amino acids selected from the group consisting of threonine, valine, tyrosine, tryptophan, aspartic acid, and serine. In certain embodiments, the composition comprises one or more free amino acids selected from the group consisting of threonine, valine, tyrosine, tryptophan, aspartic acid, serine, and derivatives thereof. In certain embodiments, the composition comprises one or more free amino acids selected from the group consisting of threonine, valine, tyrosine, tryptophan, and aspartic acid. The composition preferably comprises one or more free amino acids selected from the group consisting of threonine, valine, serine, tyrosine, and tryptophan. In certain embodiments, the composition comprises two or more free amino acids selected from the group consisting of threonine, valine, tyrosine, tryptophan, aspartic acid, and serine. In certain embodiments, the composition comprises three or more free amino acids selected from the group consisting of threonine, valine, tyrosine, tryptophan, aspartic acid, and serine. In certain embodiments, the composition comprises four or more free amino acids selected from the group consisting of threonine, valine, tyrosine, tryptophan, aspartic acid, and serine. In certain embodiments, the composition comprises threonine, valine, tyrosine, tryptophan, and aspartic acid. In certain embodiments, the composition comprises the free amino acids of threonine, valine, tyrosine, tryptophan, and serine.

These amino acids, if present in the composition, may be present in, for example, the following concentrations: threonine at about 0.4 to about 1.5, about 0.7 to about 1.3, or about 0.9 to about 1.1 grams/liter; valine at about 0.7 to about 1.7, about 0.9 to about 1.5, or about 1.1 to about 1.3 grams/liter; serine at about 0.6 to about 1.6, about 0.8 to about 1.4, about 1.0 to about 1.2 grams/liter; tyrosine at about 0.05 to about 0.4, or about 0.1 to about 0.3 grams/liter; and tryptophan at about 1.1 to about 2.1, about 1.3 to about 1.9, or about 1.5 to about 1.7 grams/liter. In a certain embodiment, the therapeutic composition comprises threonine (about 1.0 grams/liter), valine (about 1.2 grams/liter), serine (about 1.1 grams/liter), tyrosine (about 0.2 grams/liter), and tryptophan (about 1.6 grams/liter). In one embodiment the composition does not include serine.

In a further embodiment, the composition comprises, or consists essentially of only one free amino acid selected from threonine, valine, tyrosine, and tryptophan, and/or derivatives thereof. In a further embodiment, the therapeutic composition comprises, or consists essentially of threonine as a free amino acid. The therapeutic composition may also comprise, or consist essentially of, valine as the free amino acid. In addition, the therapeutic composition comprises, or consists essentially of, tyrosine as a free amino acid. The therapeutic composition comprises, or consists essentially of, tryptophan as a free amino acid. Furthermore, the therapeutic composition comprises, or consists essentially of, aspartic acid as a free amino acid. In certain embodiments, the composition comprises serine as a free amino acid.

In another embodiment, the composition may also comprise, or consist essentially of, two free amino acids selected from the group consisting of threonine, valine, serine, tyrosine, tryptophan, and aspartic acid, including the combination of threonine and valine, the combination of threonine and serine, the combination of threonine and tyrosine, the combination of threonine and tryptophan, the combination of valine and serine, the combination of valine and tyrosine, the combination of valine and tryptophan, the combination of serine and tyrosine, the combination of serine and tryptophan, the combination of tyrosine and aspartic acid, the combination of serine and aspartic acid, the combination of valine and aspartic acid, the combination of threonine and aspartic acid, the combination of tryptophan and aspartic acid, and the combination of tyrosine and tryptophan.

In another embodiment, the composition may comprise, or consist essentially of, three free amino acids selected from the group consisting of threonine, valine, serine, tyrosine, tryptophan and aspartic acid, including the combination of threonine, valine, and serine; the combination of threonine, valine, and tyrosine; the combination of threonine, valine, and tryptophan; the combination of threonine, serine, and tyrosine; the combination of threonine, serine, and tryptophan; the combination of threonine, tyrosine, and tryptophan; the combination of valine, serine, and tyrosine; the combination of valine, serine, and tryptophan; the combination of valine, tyrosine, and tryptophan; and the combination of serine, tyrosine, and tryptophan; the combination of threonine, valine, and aspartic acid, the combination of threonine, serine, and aspartic acid; the combination of threonine, tyrosine, and aspartic acid; the combination of threonine, tryptophan and aspartic acid; the combination of valine, serine, and aspartic acid; the combination of valine, tyrosine, and aspartic acid; the combination of valine, tryptophan and aspartic acid; the combination of serine, tyrosine and aspartic acid; the combination of serine, tryptophan and aspartic acid; the combination of tyrosine, tryptophan and aspartic acid.

In another embodiment, the composition may comprise, or consist essentially of, four free amino acids selected from the group consisting of threonine, valine, serine, tyrosine, tryptophan, and aspartic acid, including the combination of threonine, valine, serine, and tyrosine; the combination of threonine, valine, serine, and tryptophan; the combination of threonine, valine, tyrosine, and tryptophan; the combination of threonine, serine, tyrosine, and tryptophan; and the combination of valine, serine, tyrosine, and tryptophan; the combination of threonine, valine, serine, and aspartic acid; the combination of threonine, valine, tyrosine, and aspartic acid; the combination of threonine, valine, tryptophan, and aspartic acid; the combination of threonine, serine, tyrosine, and aspartic acid; the combination of threonine, serine, tryptophan, and aspartic acid; the combination of threonine, tyrosine, tryptophan, and aspartic acid; the combination of valine, serine, tyrosine, and aspartic acid; the combination of valine, serine, tryptophan, and aspartic acid; the combination of valine, tyrosine, tryptophan, and aspartic acid; the combination of serine, tyrosine, tryptophan, and aspartic acid.

In another embodiment, the composition may comprise, or consist essentially of, five free amino acids selected from the group consisting of threonine, valine, serine, tyrosine, tryptophan, and aspartic acid, including the combination of threonine, valine, serine, tyrosine and tryptophan; the combination of threonine, valine, serine, tyrosine, and aspartic acid; the combination of threonine, valine, serine, tryptophan, and aspartic acid; the combination of threonine, valine, tyrosine, tryptophan, and aspartic acid; the combination of threonine, serine, tyrosine, tryptophan, and aspartic acid.

In another embodiment, the composition may comprise, or consist essentially of, threonine, valine, serine, tyrosine, tryptophan, and aspartic acid as free amino acids.

In certain embodiments, the compositions may comprise natural amino acids or derivatives thereof that retain substantially the same, or better, activity in terms of enhancing the survival, proliferation, and/or development of stem cells and/or progenitor cells. In certain embodiments, the compositions may comprise natural amino acids or derivatives thereof that retain substantially the same, or better, activity in terms of wound healing, treating skin conditions (e.g., atopic dermatitis, psoriasis, bed sores, or condition related to the aging of skin), and/or improving mucosal barrier function in a subject in need thereof. The derivatives may be, for example, enantiomers, and include both the D- and L-forms of the amino acids. The derivatives may be, for example, iodotyrosine, or norvaline. Other amino acid derivatives include, for example, norleucine, ornithine, penicillamine, pyroglutamine derivatives, or other derivatives of alanine, asparagine, aspartic acid, cysteine, glutamic acid, glycine, isoleucine, leucine, lysine, methionine, proline, phenylalanine, serine, threonine, tryptophan, or valine. In certain embodiments, the amino acid derivatives are derivatives of threonine, valine, tyrosine, tryptophan, aspartic acid, or serine. Other amino acid derivatives include, but are not limited to, those that are synthesized by, for example, acylation, methylation, and/or halogenation of the amino acid. These include, for example, β-methyl amino acids, C-methyl amino acids, and N-methyl amino acids.

In some specific embodiments, the composition of the present disclosure does not comprise one or more amino acids selected from the group consisting of glycine and asparagine. Or, in certain embodiments, even if these amino acids are present in the composition, they are not present in an amount that would inhibit stem cell and/or progenitor cell proliferation and/or development.

In certain specific embodiments, the composition of the present disclosure does not include, or only comprises negligible amounts of, one or more of the free amino acids selected from the group consisting of glycine and asparagine. In further embodiments, the therapeutic composition does not include glycine as a free amino acid; and/or the therapeutic composition does not include asparagine as a free amino acid. In another specific embodiment, the composition does not include, or only includes negligible amounts of, glycine and/or asparagine. In one embodiment, the composition does not include glycine and/or asparagine; and any di-, oligo-, or polypeptides or proteins that can be hydrolyzed into glycine and/or asparagine. In one embodiment, the composition does not include glutamine and/or methionine; and any di-, oligo-, or polypeptides or proteins that can be hydrolyzed into glutamine and/or methionine.

In certain specific embodiments, the therapeutic composition may comprise lysine, wherein the total concentration of glycine, wherein the total concentration of glycine is less than 300 mg/l, 100 mg/l, 50 mg/l, 10 mg/l, 5 mg/l, 1 mg/l, 0.5 mg/l, or 0.01 mg/l. The therapeutic composition may further comprise asparagine, wherein the total concentration of asparagine is less than 10 mg/l, 5 mg/l, 1 mg/l, 0.5 mg/l, or 0.01 mg/l.

In an alternative embodiment, the composition may comprise free amino acid glutamine, and, optionally, one or more glutamine-containing di peptides, wherein the total concentration of the free amino acid glutamine and the glutamine-containing dipeptide(s) is less than 300 mg/l, or any concentrations lower than 300 mg/l, such as 100 mg/l, 50 mg/l, 10 mg/l, 5 mg/l, 1 mg/l, 0.5 mg/l, or 0.01 mg/l. In certain embodiments, the composition may comprise free amino acid glutamine, and, optionally, one or more glutamine-containing peptides, wherein the total concentration of the free amino acid glutamine and the glutamine-containing peptide(s) is less than 300 mg/l, or any concentrations lower than 300 mg/l, such as 100 mg/l, 50 mg/l, 10 mg/l, 5 mg/l, 1 mg/l, 0.5 mg/l, or 0.01 mg/l.

In another alternative embodiment, the therapeutic composition may comprise free amino acid methionine, and, optionally, one or more methionine-containing dipeptides, wherein the total concentration of the free amino acid methionine and the methionine-containing dipeptide(s) is less than 300 mg/l, or any concentrations lower than 300 mg/l, such as 100 mg/l, 50 mg/l, 10 mg/l, 5 mg/l, 1 mg/l, 0.5 mg/l, or 0.01 mg/l. In certain embodiments, the composition may comprise free amino acid methionine, and, optionally, one or more methionine-containing dipeptides, wherein the total concentration of the free amino acid methionine and the methionine-containing peptide(s) is less than 300 mg/l, or any concentrations lower than 300 mg/l, such as 100 mg/l, 50 mg/l, 10 mg/l, 5 mg/l, 1 mg/l, 0.5 mg/l, or 0.01 mg/l.

In certain embodiments, the composition also comprises additives (e.g., nutrients, electrolytes, vitamins, minerals, etc.). In certain embodiments, the composition comprises iron or zinc. In certain embodiments, the therapeutic composition comprises one or more electrolytes selected from, for example, Na⁺; K⁺; HCO₃⁻; CO₃²⁻; Ca²⁺; Mg²⁺; Fe₂; Cl⁻; phosphate ions, such as H₂PO₄⁻, HPO₄²⁻, and PO₄³⁻; zinc; iodine; copper; iron; selenium; chromium; and molybdenum. In an alternative embodiment, the composition does not contain HCO₃⁻ or CO₃²⁻. In another alternative embodiment, the composition comprises HCO₃⁻ and CO₃²⁻ at a total concentration of less than 5 mg/l, or concentrations lower than 5 mg/l. In certain embodiments, the composition does not contain electrolytes. For example, in certain embodiments, the composition does not include one or more, or any, of Na⁺; K⁺; HCO₃⁻; CO₃²⁻; Ca²⁺; Mg²⁺; Fe²; Cl⁻; phosphate ions, such as H₂PO₄⁻, HPO₄²⁻, and PO₄³⁻; zinc; iodine; copper; iron; selenium; chromium; and molybdenum.

In certain embodiments, the composition does not contain one or more of the ingredients selected from oligo-, polysaccharides, and carbohydrates; oligo- or polypeptides, or proteins; lipids; small-, medium-, and/or long-chain fatty acids; and/or food containing one or more of the above-mentioned nutrients. In certain embodiments, the composition does not include glucose or sucrose.

In one embodiment, phosphate ions, such as H₂PO₄⁻, HPO₄²⁻, and PO₄³⁻, are used to buffer the composition of the present disclosure. In one embodiment, the therapeutic composition uses HCO₃⁻ or CO₃²⁻ as a buffer. In another embodiment, the therapeutic composition does not use HCO₃⁻ or CO₃²⁻ as buffer.

In certain embodiments, the composition comprises: valine, threonine, tyrosine, electrolytes, Na+ (about 10 mmol to 60 mmol), and K+ (about 1 mmol to 20 mmol). In certain embodiments, the composition comprises a buffer.

Stem Cell and/or Progenitor Cell Therapies and Therapies for Wound Healing, Treating Skin Conditions, and/or Improving Mucosal Barrier Function

Described herein are compositions of amino acids as therapies for treating skin disorders. The present disclosure provides compositions and methods enhancing the survival, proliferation, and/or development of stem cells and/or progenitor cells. The present disclosure provides compositions and methods for treating a disease or condition that is related to mucosal barrier function, e.g., wound healing, treating skin conditions (e.g., atopic dermatitis, psoriasis, bed sores, or condition related to the aging of skin), and/or improving mucosal barrier function in a subject in need thereof. The number of stem cells and/or progenitor cells can be increased by increasing survival, proliferation, and/or development of the cells. In one embodiment, the method comprises exposing the stem cells and/or the progenitor cells to a composition of the present disclosure. The stem cells and/or the progenitor cells can be exposed to the composition in culture, ex vivo, in situ, or in vivo, including after being administered, implanted, or delivered into a subject.

The subject can be, for example, a human in which promoting the survival, proliferation and/or development of stem cells and/or progenitor cells is needed. The subject can be, for example, a human subject with a disease or condition in need of treatment. In addition to humans the animal can be of any species, including, but not limited to, mammalian species including, but not limited to, domesticated and laboratory animals such as rabbits, dogs, cats, mice, rats, guinea pigs, and hamsters; livestock such as horses, cattle, pigs, sheep, goats, ducks, geese, and chickens; other primates such as apes, chimpanzees, orangutans, and monkeys; fish; amphibians such as frogs and salamanders; reptiles such as snakes and lizards; and other animals such as fox, camels, bears, antelopes, llamas, weasels, mink, beavers, ermines, otters, sable, seals, coyotes, chinchillas, deer, muskrats, and possum.

In one embodiment, the methods lead to an increase in the survival, proliferation, and/or development of stem cells and/or the progenitor cells. In certain embodiments, the methods lead to an improvement in the condition of a subject with a disease or conditions that is related to mucosal barrier function, e.g., wounds, skin conditions (e.g., atopic dermatitis, psoriasis, bed sores, or condition related to the aging of skin), and/or mucosal barrier function.

In one embodiment, the method comprises introducing the composition according to the present invention to stem and/or progenitor cells in culture for promoting survival, proliferation, and/or development. The composition may thus be used to obtain enhanced quantities of the cells for use in treating various diseases and conditions.

In one embodiment, the present disclosure provides a method of improving therapeutic outcomes of implanted stem cells comprising administering a composition of the present disclosure in conjunction with stem cell implantation. The administration of the composition can be at, or proximate to, a target stem cell implantation site in a human or nonhuman animal. In certain embodiments, provided herein is a method for treating a disease or conditions that is related to mucosal barrier function, e.g., wound healing, treating skin conditions (e.g., atopic dermatitis, psoriasis, bed sores, or condition related to the aging of skin), and/or improving mucosal barrier function in a subject in need thereof, the method comprising administering a composition described herein to the subject in need thereof.

In one embodiment, recipients of administered stem and/or progenitor cells can be immunosuppressed, either through the use of immunosuppressive drugs such as cyclosporin, or through local immunosuppression strategies employing locally applied immunosuppressants, but such immunosuppression need not necessarily be a prerequisite in certain immuneprivileged tissues such as, for example, brain and eye tissues.

In certain embodiments, administered stem and/or progenitor cells are autologous in nature, i.e., prepared from the recipient's own tissue. In such instances, the progeny of stem cells can be generated from dissociated or isolated tissue and proliferated in vitro using the composition of the present disclosure. Upon suitable expansion of cell numbers, the cells can be harvested and readied for administration into the recipient's affected tissue.

In one embodiment, the present disclosure provides a method for promoting the proliferation and differentiation of stem cells in a subject in such need, wherein said method comprises: identifying a subject in such need, and administering, to the subject, an effective amount of a composition comprising, consisting essentially of, or consisting of one or more free amino acids selected from the group consisting of threonine, valine, tyrosine, tryptophan, aspartic acid and serine; and optionally, one or more pharmaceutically acceptable carriers, adjuvants and/or other active agents, wherein the composition has a total osmolarity from about 100 to about 280 mosm and a pH of about 2.5 to about 6.5. Alternatively, an effective amount of a composition comprising, consisting essentially of, or consisting of valine and serine and one or more free amino acids comprising tyrosine, aspartic acid, or threonine, tryptophan; and optionally, one or more pharmaceutically acceptable carriers, adjuvants and/or other active agents is used, wherein the composition has a total osmolarity from about 100 to about 280 mosm and a pH of about 2.5 to about 6.5. In another alternative embodiment, an effective amount of a composition comprising, consisting essentially of, or consisting of valine and serine and one or more free amino acids comprising tyrosine, aspartic acid, threonine, tryptophan, lysine, or isoleucine; and optionally, one or more pharmaceutically acceptable carriers, adjuvants and/or other active agents is used, wherein the composition has a total osmolarity from about 100 to about 280 mosm and a pH of about 2.5 to about 6.5. In certain embodiments, provided herein is a method for treating a disease or conditions that is related to mucosal barrier function, e.g., wound healing, treating skin conditions (e.g., atopic dermatitis, psoriasis, bed sores, or condition related to the aging of skin), and/or improving mucosal barrier function, wherein said method comprises: identifying a subject in such need, and administering, to the subject, an effective amount of a composition comprising, consisting essentially of, or consisting of one or more free amino acids selected from the group consisting of threonine, valine, tyrosine, tryptophan, aspartic acid and serine; and optionally, one or more pharmaceutically acceptable carriers, adjuvants and/or other active agents, wherein the composition has a total osmolarity from about 100 to about 280 mosm and a pH of about 2.5 to about 6.5. Alternatively, an effective amount of a composition comprising, consisting essentially of, or consisting of valine and serine and one or more free amino acids comprising tyrosine, aspartic acid, or threonine, tryptophan; and optionally, one or more pharmaceutically acceptable carriers, adjuvants and/or other active agents is used, wherein the composition has a total osmolarity from about 100 to about 280 mosm and a pH of about 2.5 to about 6.5. In another alternative embodiment, an effective amount of a composition comprising, consisting essentially of, or consisting of valine and serine and one or more free amino acids comprising tyrosine, aspartic acid, threonine, tryptophan, lysine, or isoleucine; and optionally, one or more pharmaceutically acceptable carriers, adjuvants and/or other active agents is used, wherein the composition has a total osmolarity from about 100 to about 280 mosm and a pH of about 2.5 to about 6.5.

In some embodiments, the present disclosure can be used to promote the proliferation and differentiation of stem cells in viral, fungal, or bacterial infection-induced conditions and diseases, for example, viral, fungal, or bacterial infection-induced bone marrow suppression. For example, the compositions and methods can be used to treat a patient with a low platelet count caused by, for example, the Dengue virus.

The compositions described herein can also be used to treat, or ameliorate the symptoms of, for example, deficits caused by a neurodegenerative disease, traumatic injury, neurotoxic injury, ischemia, developmental disorders, disorders affecting vision, injuries or disease of the spinal cord, demyelinating diseases, autoimmune diseases, infections, inflammatory diseases, or corporal diseases.

In certain embodiments, implanted stem cells are capable of proliferating, migrating to an area of tissue damage, and/or differentiating in a tissue-specific manner and functioning in a manner that reduces the deficit.

In one embodiment, the method and composition according to the present disclosure is particularly useful for patients that are exposed to radiation, or receive radiation, chemo-, and/or proton therapy.

The compositions of the present disclosure can be used in the treatment or amelioration of any diseases or conditions in need of proliferation and/or development of stem and/or progenitor cells.

Additionally, the present disclosure can be used to promote proliferation of stem cells for the treatment or amelioration of injury caused by chemotherapeutic agents including, but not limited to, cisplatin, 5-fluorouracil (5-FU), hydroxyurea, etoposide, arabinoside, 6-mercaptopurine, 6-thioguanine, fludarabine, methothexate, steroids, and/or a combination thereof. Exemplary chemotherapeutic agents include, but are not limited to, anti-estrogens (e.g. tamoxifen, raloxifene, and megestrol), LHRH agonists (e.g. goscrclin and leuprolide), anti-androgens (e.g. flutamide and bicalutamide), photodynamic therapies (e.g. vertoporfin (BPD-MA), phthalocyanine, photosensitizer Pc4, and demethoxy-hypocrellin A (2BA-2-DMHA)), nitrogen mustards (e.g. cyclophosphamide, ifosfamide, trofosfamide, chlorambucil, estramustine, and melphalan), nitrosoureas (e.g. carmustine (BCNU) and lomustine (CCNU)), alkylsulphonates (e.g. busulfan and treosulfan), triazenes (e.g. dacarbazine, temozolomide), platinum containing compounds (e.g. cisplatin, carboplatin, oxaliplatin), vinca alkaloids (e.g. vincristine, vinblastine, vindesine, and vinorelbine), taxoids (e.g. paclitaxel or a paclitaxel equivalent such as nanoparticle albumin-bound paclitaxel (ABRAXANE), docosahexaenoic acid bound-paclitaxel (DHA-paclitaxel, Taxoprexin), polyglutamate bound-paclitaxel (PG-paclitaxel, paclitaxel poliglumex, CT-2103, XYOTAX), the tumor-activated prodrug (TAP) ANG1005 (Angiopep-2 bound to three molecules of paclitaxel), paclitaxel-EC-1 (paclitaxel bound to the erbB2-recognizing peptide EC-1), and glucose-conjugated paclitaxel, e.g., 2′-paclitaxel methyl 2-glucopyranosyl succinate; docetaxel, taxol), epipodophyllins (e.g. etoposide, etoposide phosphate, teniposide, topotecan, 9-aminocamptothecin, camptoirinotecan, irinotecan, crisnatol, mytomycin C), anti-metabolites, DHFR inhibitors (e.g. methotrexate, dichloromethotrexate, trimetrexate, edatrexate), IMP dehydrogenase inhibitors (e.g. mycophenolic acid, tiazofurin, ribavirin, and EICAR), ribonuclotide reductase inhibitors (e.g. hydroxyurea and deferoxamine), uracil analogs (e.g. 5-fluorouracil (5-FU), floxuridine, doxifluridine, ratitrexed, tegafur-uracil, capecitabine), cytosine analogs (e.g. cytarabine (ara C), cytosine arabinoside, and fludarabine), purine analogs (e.g. mercaptopurine and Thioguanine), Vitamin D3 analogs (e.g. EB 1089, CB 1093, and KH 1060), isoprenylation inhibitors (e.g. lovastatin), dopaminergic neurotoxins (e.g. 1-methyl-4-phenylpyridinium ion), cell cycle inhibitors (e.g. staurosporine), actinomycin (e.g. actinomycin D, dactinomycin), bleomycin (e.g. bleomycin A2, bleomycin B2, peplomycin), anthracycline (e.g. daunorubicin, doxorubicin, pegylated liposomal doxorubicin, idarubicin, epirubicin, pirarubicin, zorubicin, mitoxantrone), MDR inhibitors (e.g. verapamil), Ca²⁺ ATPase inhibitors (e.g. thapsigargin), imatinib, thalidomide, lenalidomide, tyrosine kinase inhibitors (e.g., axitinib (AG013736), bosutinib (SKI-606), cediranib (RECENTIN™, AZD2171), dasatinib (SPRYCEL®, BMS-354825), erlotinib (TARCEVA®), gefitinib (IRESSA®), imatinib (Gleevec®, CGP57148B, STI-571), lapatinib (TYKERB®, TYVERB®), lestaurtinib (CEP-701), neratinib (HKI-272), nilotinib (TASIGNA®), semaxanib (semaxinib, SU5416), sunitinib (SUTENT®, SU11248), toceranib (PALLADIA®), vandetanib (ZACTIMA®, ZD6474), vatalanib (PTK787, PTK/ZK), trastuzumab (HERCEPTIN®), bevacizumab (AVASTIN®), rituximab (RITUXAN®), cetuximab (ERBITUX®), panitumumab (VECTIBIX®), ranibizumab (Lucentis®), nilotinib (TASIGNA®), sorafenib (NEXAVAR®), everolimus (AFINITOR®), alemtuzumab (CAMPATH®), gemtuzumab ozogamicin (MYLOTARG®), temsirolimus (TORISEL®), ENMD-2076, PCI-32765, AC220, dovitinib lactate (TKI258, CHIR-258), BIBW 2992 (TOVOK™), SGX523, PF-04217903, PF-02341066, PF-299804, BMS-777607, ABT-869, MP470, BIBF 1120 (VARGATEF®), AP24534, JNJ-26483327, MGCD265, DCC-2036, BMS-690154, CEP-11981, tivozanib (AV-951), OSI-930, MM-121, XL-184, XL-647, and/or XL228), proteasome inhibitors (e.g., bortezomib (VELCADE)), mTOR inhibitors (e.g., rapamycin, temsirolimus (CCI-779), everolimus (RAD-001), ridaforolimus, AP23573 (Ariad), AZD8055 (AstraZeneca), BEZ235 (Novartis), BGT226 (Norvartis), XL765 (Sanofi Aventis), PF-4691502 (Pfizer), GDC0980 (Genetech), SF1126 (Semafoe) and OSI-027 (OSI)), oblimersen, gemcitabine, carminomycin, leucovorin, pemetrexed, cyclophosphamide, dacarbazine, procarbizine, prednisolone, dexamethasone, campathecin, plicamycin, asparaginase, aminopterin, methopterin, porfiromycin, melphalan, leurosidine, leurosine, chlorambucil, trabectedin, procarbazine, discodermolide, carminomycin, aminopterin, and hexamethyl melamine.

In one embodiment, the stem cells and/or progenitor cells have been subjected to radiation prior to treatment with the composition of the present disclosure. In another embodiment, the stem cells and/or progenitor cells will be subjected to radiation after treatment with the composition of the present disclosure. The radiation may be administered to the cells, for example, 1 minute, 5 minutes, 30 minutes, 1 hour, 6 hours, 12 hours, 1 day, 5 days, 7 days, 14 days, 30 days, 60 days, 3 months, 6 months, 1 year, 2 years, or 3 years or more, before or after treatment of the cells with the composition described herein. The dose of radiation may be, for example, at least 1, 2, 3, 4, 5, 6, 7, 8, 9, 10, 15, 20, 25, 30, 35, 40, 45, 50, 55, 60, 65, 70, 80, 85, 90, 95, 100, 120, or 150 Gy.

In one embodiment, the present disclosure can be used in the context of bone marrow transplants. Bone marrow stem and/or progenitor cells can be treated in vivo or ex vivo with a composition of the present disclosure. Such treatment enhances the survival, proliferation, and/or development of the cells.

In another embodiment, the present disclosure can be used in the context of Prochymal, which is used in the management of acute graft-vs-host disease in children. It is an allogenic stem therapy based on mesenchymal stem cells (MSCs) derived from the bone marrow of adult donors. The survival, proliferation, and/or development of the MSCs can be enhanced by contacting them, either in vivo or ex vivo with the composition of the present disclosure.

In another embodiment, the compositions and methods of the present disclosure can be used in cardiac treatments. Stem-cell therapy for treatment of myocardial infarction often makes use of autologous bone-marrow stem cells; however, other types of adult stem cells may be used, such as adipose tissue-derived stem cells. In one embodiment, use of stem cell therapy results in cardiac tissue regeneration to reverse the tissue loss underlying the development of heart failure after cardiac injury.

In another embodiment, the compositions and methods of the present disclosure can be used in blood cell formation and expansion. Fully mature human red blood cells may be generated ex vivo by hematopoietic stem cells (HSCs), which are precursors of red blood cells. In this process, HSCs can be grown together with stromal cells, creating an environment that mimics the conditions of bone marrow, the natural site of red-blood-cell growth. In addition to using compositions of the present disclosure, erythropoietin, a growth factor, can be added, coaxing the stem cells to complete terminal differentiation into red blood cells. The compositions and methods can also be used to expand populations of red blood cells, white blood cells, and/or platelets to improve, for example, oxygen carrying capacity (such as for athletes), the immune system (including for treating immune-compromised subjects), and for improving clotting.

In another embodiment, cochlear hair can be re-grown using embryonic stem cells treated with the compositions of the present disclosure.

In another embodiment, stem cells treated according to the present disclosure can be used to treat blindness and vision impairment. In a specific embodiment, the compositions and methods are used to treat cornea laceration.

In another embodiment, the present disclosure can be used in the context of enhancing the success of tissue transplantations, including the transplantation of insulin-producing pancreatic beta cells. These cells can be prepared from, for example, embryonic stem cells that have been caused to differentiate into the beta cells. These cells may be treated in vivo or ex vivo with the compositions of the present disclosure.

In another aspect, the present disclosure provides methods of treating a wound and/or promoting wound healing in a subject in need thereof, the method comprising administering to the subject a composition described herein. In certain embodiments, the present disclosure provides methods of treating a wound in a subject in need thereof, the method comprising administering to the subject a composition described herein. In certain embodiments, the present disclosure provides methods of treating a wound or burn in a subject in need thereof, the method comprising administering to the subject a composition described herein. In certain embodiments, the present disclosure provides methods of treating a burn in a subject in need thereof, the method comprising administering to the subject a composition described herein. In certain embodiments, the wound is a partial thickness or full thickness wound. In certain embodiments, the burn is a partial thickness or full thickness burn.

The present disclosure can also be utilized in the context of wound healing. In an adult, wounded tissue is most often replaced by scar tissue, which is characterized by disorganized collagen structure, loss of hair follicles, and irregular vascular structure. In one embodiment, stem cell “seeds” are placed inside a tissue bed in a wound bed and allowing the stem cells to stimulate differentiation in the tissue bed cells. This method can be greatly enhanced by contacting the wound, with or without the addition of stem cells, with a composition of the present disclosure. In certain embodiments, the composition is applied to the skin. In certain embodiments, the composition is applied to stem cells and/or progenitor cells.

In other embodiments, the composition and methods described herein are useful for cosmetic applications where, for example, rejuvenation of the various layers of the skin and/or the underlying tissues is desired. This rejuvenation can be aided by, for example, the enhanced survival, proliferation, and/or development of stem cells and/or progenitor cells. This rejuvenation can be aided by, for example, by treating a disease or conditions that is related to mucosal barrier function, e.g., wounds, skin conditions (e.g., atopic dermatitis, psoriasis, bed sores, or condition related to the aging of skin), and/or mucosal barrier function.

In this embodiment, the methods of the present disclosure generally include the step of topically applying the compositions to the skin (e.g., epidermis) of the patient needing such treatment, wherein a therapeutically effective amount of such composition is applied. In one embodiment, the composition is applied to the face.

Advantageously, the present invention provides compositions and methods that combat the aging of skin, wherein combating the aging of skin can include, for example, treating the appearance of wrinkles, fine lines, and other forms of undesirable skin texture. By presenting the composition to the dermal and/or epidermal layer(s) of the skin, the form, strength, as well as function of the skin is enhanced. In certain embodiments, the composition and methods described herein are useful for beauty applications where, for example, rejuvenation of the various layers of the skin and/or the underlying tissues is desired.

In another aspect, the present disclosure provides methods of treating and/or preventing a skin condition (e.g., atopic dermatitis, psoriasis, or condition related to the aging of skin) in a subject in need thereof, the method comprising administering to the subject a composition described herein. In certain embodiments, the skin condition is atopic dermatitis, psoriasis, the aging of skin, a condition related to the aging of skin, or bed sores. In some embodiments, the skin condition is pruritus (itch), psoriasis, eczema, burns, or dermatitis. In certain embodiments, the skin condition is psoriasis. In certain embodiments, the skin condition is pruritis.

In certain embodiments, the compositions of the present disclosure comprise agents, in addition to the amino acids, that are useful in delaying, minimizing, or eliminating skin aging, wrinkling, and/or other histological changes typically associated with the intrinsic conditions (such as aging, menopause, acne, etc.) and extrinsic conditions (such as environmental pollution, wind, heat, sunlight, radiation, low humidity, harsh surfactants, etc.).

The present invention is useful for therapeutically and/or prophylactically improving visible and/or tactile characteristics in skin. For example, in one embodiment, the length, depth, and/or other dimension of lines and/or wrinkles are decreased.

In one embodiment, the composition applied to the skin or other tissue can further comprise collagen and/or hyaluronic acid (HA). In one embodiment, the HA is cross-linked HA. The composition can further comprise components such as, but not limited to, dermatologically acceptable carriers, desquamation agents, anti-acne agents, anti-wrinkle agents/anti-atrophy agents, vitamin B3 compounds, retinoids, hydroxyl acids, anti-oxidants/Radical scavengers, chelators, flavonoids, anti-inflammatory agents, anti-cellulite agents, topical anesthetics, tanning agents, skin lightening agents, skin soothing and skin healing agents, antimicrobial and antifungal agents, sunscreen agents, conditioning agents, structuring agents, thickening agent (including thickeners and gelling agents), composition preparation and preservatives. In this regard, international PCT application publication, WO 2008/089408 is incorporated herein, by reference, in its entirety.

The composition of the present disclosure can also be administered at a surgical site, including at a site of minimally invasive surgery, to improve healing and the surgical outcome.

Stem cells can also be used, in accordance with the present disclosure to treat infertility. In certain embodiments, a person is first diagnosed with a condition for which stem cell survival, proliferation and/or development would be beneficial. For example, the subject may be diagnosed with the condition and the composition of the subject application is then administration via a route, and in an amount, that results in stem cell survival, proliferation and/or development. Preferably, such administration then results in treatment (e.g., an improvement) of the condition.

Use of the Composition to Promote Stem and/or Progenitor Cell Proliferation and/or Development, and to Treat a Disease or Conditions Related to Mucosal Barrier Function, Skin Conditions, and Improve Mucosal Barrier Function

Described herein are uses of compositions of amino acids for treating, e.g., skin disorders. In one aspect thereof, methods are presented for treating a disease or conditions that are related to mucosal barrier function, e.g., wound healing, treating skin conditions (e.g., atopic dermatitis, psoriasis, bed sores, or condition related to the aging of skin), and/or a condition related to improving mucosal barrier function in a subject in need thereof.

### Formulations and Kits

The present disclosure provides for therapeutic or pharmaceutical compositions comprising a therapeutically effective amount of the subject composition and, optionally, one or more pharmaceutically acceptable carriers. The present disclosure provides for therapeutic, pharmaceutical, cosmetic, or nutritional compositions comprising a therapeutically effective amount of the subject composition and, optionally, one or more pharmaceutically acceptable carriers. Such pharmaceutical carriers can be liquids, such as water. The therapeutic composition can also comprise excipients, adjuvants, flavoring agents, etc. that facilitate processing of the active compounds into preparations that can be used pharmaceutically. Proper formulation is dependent upon the route of administration chosen. In an embodiment, the therapeutic composition and all ingredients contained therein are sterile. Examples of suitable pharmaceutical carriers are described in “Remington's Pharmaceutical Sciences” by E. W. Martin. Such compositions contain a therapeutically effective amount of the therapeutic composition, together with a suitable amount of carrier so as to provide the form for proper administration to the patient. The formulation should suit the enteral mode of administration.

In one embodiment, the administration of the composition can be systemic. Oral, intravenous, intra-arterial, subcutaneous, intra-peritoneal, intra-muscular, intra-ventricular, intranasal, transmucosal, subcutaneous, topical, rectal, and other modes of administration are all contemplated.

In one embodiment, for injection, the active ingredient can be formulated in aqueous solutions, preferably in physiologically compatible buffers. For transmucosal administration, penetrants appropriate to the barrier to be permeated are used in the formulation. Such penetrants are generally known in the art. For oral administration, the active ingredient can be combined with carriers suitable for inclusion into tablets, pills, dragees, capsules, liquids, gels, syrups, slurries, suspensions and the like. Formulations can also be prepared for use in inhalation therapy. For administration by inhalation, the composition can be delivered in the form of an aerosol spray presentation from pressurized packs or a nebulizer, with the use of a suitable propellant. The composition can also be administered via inhalation or other route as a powder.

Therapeutically effective doses of the presently described composition can be determined by one of skill in the art, with a goal of achieving a desired number of stem cells and/or precursor cells. An increase in the number of stem cells and precursor cells can be assessed using markers of these cells, or by determining an increase in the number of differentiated progeny of these cells. Method for measuring increased numbers of differentiated cells are known in the art. For example, immunohistochemistry, behavioral assessments or electrophysiological techniques can also be utilized. One of skill in the art can readily detect an increase in the number of cells of a specific phenotype.

In particular embodiments, the methods according to the present disclosure include administering the therapeutic composition by sustained-release systems. Suitable examples of sustained-release systems include suitable polymeric materials (such as, semi-permeable polymer matrices in the form of shaped articles, for example films, or microcapsules), suitable hydrophobic materials (for example as an emulsion in an acceptable oil) or ion exchange resins, and sparingly soluble derivatives (such as, for example, a sparingly soluble salt). Sustained-release compositions can be administered orally, parenterally, intracistemally, intraperitoneally, topically (as by powders, ointments, gels, drops or transdermal patch), or as an oral or nasal spray. Sustained-release matrices include polylactides, copolymers of L-glutamic acid and gamma-ethyl-L-glutamate, poly(2-hydroxyethyl methacrylate), ethylene vinyl acetate, or poly-D-(−)-3-hydroxybutyric acid.

In one embodiment, implantable drug infusion devices may be used to provide patients with a constant and long-term dosage or infusion of a therapeutic composition. Such device can be categorized as either active or passive.

In one embodiment, polymers can be used for ion-controlled release. Various degradable and nondegradable polymeric matrices for use in controlled drug delivery are known in the art. For example, the block copolymer, polaxamer 407, hydroxyapatite, and liposomes.

The pharmaceutical composition of the present invention may be used either alone or in combination with one or more drugs known to be effective for treating diseases. The compositions can also be formulated in combination with at least one other agent, such as stabilizing or buffer compounds, which can be administered in any sterile, biocompatible pharmaceutical carrier, including, but not limited to, saline, buffered saline, dextrose, and water. In addition to the critical components of compositions discussed herein, cells or influencing factors, the compositions can contain suitable pharmaceutically acceptable carriers comprising excipients and auxiliaries that facilitate processing of the active compounds into preparations that can be used pharmaceutically. The composition may be prepared as a single-dosage form using a pharmaceutically acceptable carrier or excipient or may be contained in a multiple-dosage container.

In one embodiment, the composition may further contain other proliferation and/or differentiation inducing agents. The proliferation or differentiation inducing agent may be any one known as a proliferation or differentiation inducing agent. Examples include fibroblast growth factor (FGF), epidermal growth factor (EGF), and retinoic acid.

The composition may further contain other commonly used additives such as an anti-oxidant, a buffer, a bacteriostat, etc., and may be formulated into an injectable formulation such as aqueous solution, suspension, emulsion, etc. a pill, a capsule, a granule, a tablet, etc., by further adding a diluent, a dispersant, a surfactant, a binder, a lubricant, etc.

Also encompassed by the disclosure are kits (e.g., pharmaceutical, therapeutic, cosmetic, or nutritional packs). The kits provided may comprise a pharmaceutical composition or compound described herein and a container (e.g., a vial, ampule, bottle, syringe, and/or dispenser package, or other suitable container). In some embodiments, provided kits may optionally further include a second container comprising a pharmaceutical excipient for dilution or suspension of a pharmaceutical composition or compound described herein. In some embodiments, the pharmaceutical composition or compound described herein provided in the first container and the second container are combined to form one unit dosage form.

Thus, in one aspect, provided are kits including a first container comprising a composition described herein. In certain embodiments, the kits are useful for treating a disorder (e.g., a skin disorder) in a subject in need thereof. In certain embodiments, the kits are useful for preventing a disorder (e.g., a skin disorder) in a subject in need thereof.

In certain embodiments, a kit described herein further includes instructions for using the composition included in the kit. A kit described herein may also include information as required by a regulatory agency such as the U.S. Food and Drug Administration (FDA). In certain embodiments, the information included in the kits is prescribing information. In certain embodiments, the kits and instructions provide for treating and/or preventing a disorder (e.g., a skin disorder) in a subject in need thereof. A kit described herein may include one or more additional pharmaceutical or other agents described herein as a separate composition.

### Methods of Administration

In one embodiment, the present disclosure involves the administration of the composition according to the present disclosure to a subject and further administering stem and/or progenitor cells to the subject. The composition is administered at a locus in said subject so as to allow contact with the cells. This may be at the same location, proximate to the location or distal to the location of where stem cells are administered.

Stem and/or progenitor cells may be administered by, for example, injecting one or a plurality of cells with a syringe, inserting the stem cells with a catheter or surgically implanting the stem cells. In certain embodiments, the stem cells are administered into a body cavity fluidly connected to a target tissue. In certain preferred embodiments, the body cavity is a brain ventricle. In other embodiments, the cells are inserted using a syringe or catheter, or surgically implanted directly at the target tissue site. In other embodiments, the stem and/or progenitor cells are administered parenterally. Parenteral administration is defined as administration via a route that bypasses the gastrointestinal tract. Parenteral administration includes intraventricular administration.

Generally compositions can be administered by any of a number of routes including, but not limited to, oral, intravenous, intramuscular, intra-arterial, intramedullary, intrathecal, intraventricular, transdermal, subcutaneous, intraperitoneal, intranasal, parenteral, topical, sublingual, or rectal means. Factors can be administered at the same location as administered stem cells. Administration of influencing factors and stem cells can be conducted simultaneously, or one prior to the other, and at the same or different locations so long as the relative locations and timing allow for the factors to influence the stem and/or progenitor cells.

For instance, by using “consisting essentially of,” the therapeutic composition does not contain any unspecified ingredients including, but not limited to, free amino acids, di-, oligo-, or polypeptides or proteins; and mono-, di-, oligo-, polysaccharides, and carbohydrates that have a direct beneficial or adverse therapeutic effect on promoting stem cell development. Also, by using the term “consisting essentially of,” the composition may comprise substances that do not have therapeutic effects on promoting stem cell development; such ingredients include carriers, excipients, adjuvants, flavoring agents, etc. that do not affect the promotion and/or development of stem cells.

It should be understood that the examples and embodiments described herein are for illustrative purposes only and that various modifications or changes in light thereof will be suggested to persons skilled in the art and are to be included within the spirit and purview of this application.

### EXAMPLES

### Example 1: Transepithelial Electrical Resistance (TEER) and Short Circuit Current (Isc) Analysis

### Primary Human Keratinocyte Isolation and Cell Culture

Primary Human keratinocytes (PHK) were isolated from neonatal foreskin. Briefly, the epidermis was separated from the dermis mechanically (pulling), placed in 3 mL trypsin (0.05%), ripped into small pieces and incubated for 5 min at 37° C. Epidermis-containing trypsin solution was passed through a 1 mL pipette repeatedly for several minutes to extract keratinocytes from the remaining tissue. After blocking of the extraction with 5 mL Dulbecco's modified Eagle's medium (DMEM) containing 10% FBS, the cell suspension was passed through a 40 μm cell strainer (BD Falcon, Franklin Lakes, N.J.) into 50 mL tubes, and centrifuged at 1,000 RPM for 5 minutes at room temperature. The PHK cell suspension diluted in serum-free low-calcium keratinocyte growth media (SFM; 6 mL) was cultured in Corning™ cell culture flasks (T25; 25 cm2; #430168) at 37° C. in a humidified incubator gassed with 95% 02 and 5% CO2 until 60% confluency (3-6 days). Media was changed on day 3, and then every 48 hours. After keratinocytes grew to the desired concentration (60% confluency), cells were trypsinized, washed and plated at a concentration of 1:1 for a 2. passage.

At passage 3 (P3), PHK were trypsinized, washed and plated on Corning™ snapwell culture inserts (0.4 μm-pore size, polyester membrane, surface area=1.12 cm², Costar #3704) and grown in low calcium keratinocyte growth media that was added to the top of the filters (200 uL), and to the bottom of each well (2 mL) until 80% confluency (ca. 6-8 days). To induce differentiation, keratinocyte growth-media was replaced by serum free, high calcium DMEM media (differentiation media) containing 1% penicillin/streptomycin, 0.2% amphotericin B. Differentiation media was changed every 48 hours until keratinocytes are fully differentiated (120 hours post differentiation).

Fully differentiated keratinocyte cell cultures (120 h post differentiation, P3) grown on snapwell culture inserts were used to analyze the effect of short-term incubation with single amino acids and amino acid combinations on transepithelial electrical resistance (TEER) and short circuit current (Isc) in Ussing chambers. Briefly, culture media was removed from the top and bottom of each filter and washed with Ca- and Mg-free PBS to remove media residues. Designated amino acid solutions were added to the top of the filters (2004) and to the bottom of the wells (2 mL), and cells were incubated for 1 hour in a humidified incubator at 37° C. gassed with 95% O₂ and 5% CO₂. After incubation, amino acids solutions were removed, and cell culture filters were immediately used for Ussing chamber experiments.

### Ussing Chamber Setup

Ussing chamber equipment for cell culture: EM-CSYS-8 Ussing Chamber System, P2300 Chambers, P2302 Sliders, VCC MC8 Multichannel Voltage/current Clamp, P2020 Electrodes, and DM MC6 Single Channel Electrode Input Module and Dummy Membranes (Physiologic Instruments, San Diego, Calif.).

Electrodes: Silver/silver chloride (Ag/AgCl) electrodes placed in 4% agar-Ringer buffer-containing electrode tips.

Ringer solution: 115 mM NaCl, 25 mM NaHCO3-, 2.4 mM K2HPO4, 0.4 mM KH2PO4, 1.2 mM MgCl2, 1.2 mM CaCl2, and 20 mM HEPES. NaOH to adjust pH to 7.4. Osmolarity 290-300 mOsm.

Protocol:
P2300 chambers were mounted in the EM-CSYS-8 Ussing chamber system;
P2302 sliders were inserted with snapwell filter dummies, and Ag/AgCl electrodes were placed into chambers;
Ag/AgCl electrodes were connected with the VCC MC8 multichannel voltage/current clamp for measuring transepithelial voltage, and current was passed;
The Ussing chamber system was pre-warmed to 37° C. with a connected circulating water bath, and 5 mL Ringer solution was added to both reservoirs of each chamber;
95% O₂ and 5% CO₂ supply was connected to the chambers, which allowed adequate oxygenation and mixed the solution within the reservoirs continuously;
Electrodes were calibrated to 0 mV at clamped voltage mode;
Dummies replaced by snap well filters with cell cultures, sliders were mounted in Ussing Chambers, and 5 mL of pre-warmed ringer solution was added to each chamber reservoir;
Cell cultures were maintained in Ringer solution at 37° C., and culture were continuously oxygenized with 95% O₂ and 5% CO₂;
Cells were equilibrated for 10-15 min;
TEER and Isc were recorded after 30 min, 60 min and 90 min.
   FIGS. 1A-1C report the relative TEER for the 30 minute time point (FIG. 1A), 60 minute time point (FIG. 1B) and 90 minute time point (FIG. 1C) in comparison to 0 min (Mean±SEM; n=4) following incubation with buffer, single amino acids or the amino acid combinations Skin 6AA (valine, threonine, tyrosine, serine, aspartic acid, tryptophan), Skin 8AA (valine, threonine, tyrosine, serine, aspartic acid, tryptophan, lysine, isoleucine) or 5AA (valine, threonine, tyrosine, serine, aspartic acid). These data are used to evaluate the tightness of the cellular junctions and the potential for improvement of the barrier function of the cellular network. Higher resistance values are interpreted as an increase in barrier function, or the tightness of the intercellular junctions.

### Example 2: Clonogenic Assay

### Cell Line and Culture Conditions

The following human cell lines were used: HFL-1 (Human Lung Fibroblasts-1), and HDFn (Human Dermal Fibroblasts); (ATCC; Manassas, USA). HFL-1 cells were cultured in F-12K Medium (Kaighn's Modification of Ham's F-12 Medium) supplemented with 10% fetal bovine serum (FBS) and 10 mg/mL penicillin/streptomycin at 37° C. in a humidified incubator gassed with 95% O₂ and 5% CO₂. HDFn cells were cultured in fibroblast basal media (ATCC) supplemented with 5 ng/mL FGFb, 7.5 mM L-Glutamine, 50 mg/mL Ascorbic acid, 1 mg/mL hydrocortisone, 5 mg/mL insulin and 2% FBS (Sigma Aldrich, St. Louis, Mo., USA). Cell concentrations in the culture were adjusted to allow exponential growth.

### Plating Density

Cells were plated in 6-well, flat bottom culture plates (Denville Scientific Inc.) at a density of 500 cells/well to allow formation of single colonies. After 24 hours, cells were incubated with single amino acids or the amino acid combinations 5AA (valine, threonine, tyrosine, serine, aspartic acid) or 8AA (valine, threonine, tyrosine, serine, aspartic acid, lysine, isoleucine, glycine) for 1 hour or 4 hours, and then maintained in fibroblast basal media for 14 days.

### Colony Evaluation

After 14 days, media was removed and cells were rinsed with PBS. Colonies were fixed and stained for 30 min in 0.5% crystal violet diluted in 50/50 methanol/water. Dishes were rinsed with water and left to dry at room temperature.

Positive colonies (>50 cells/colony) were counted by Image J software. Survival fraction (SF) was calculated as the plating efficiency of the amino acid (PA_{AA}) divided by the plating efficiency of the control (ringer; PAR) using the formula SF=PA_{AA}/PAR. Plating efficiency is calculated as the number of colonies divided by the number of plated cells using the formula: PA=colony/cells. The results are based on three repeats.

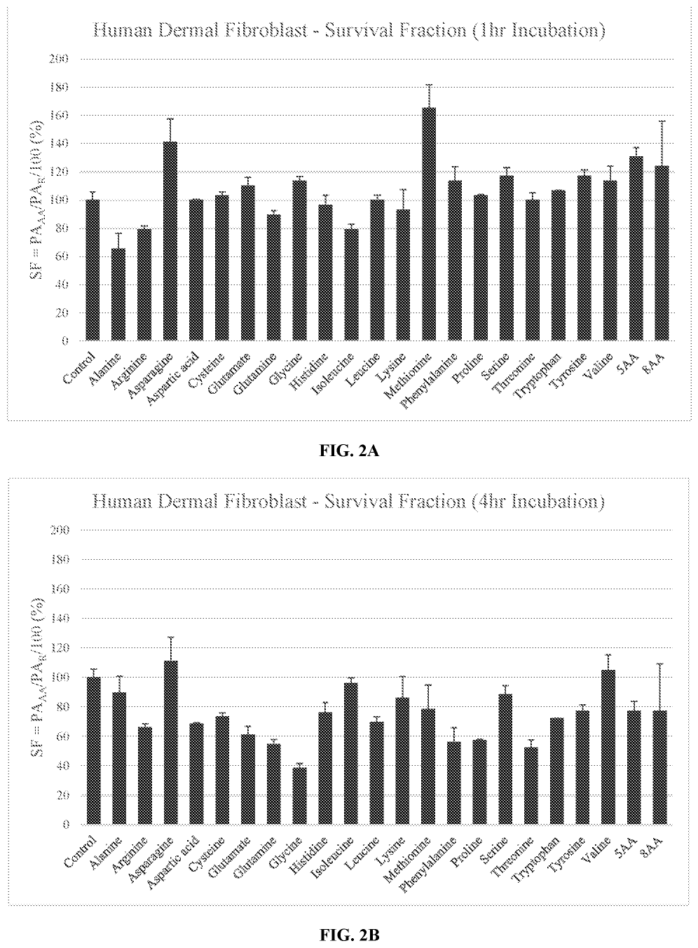

Results from the 1 hour incubation are reported in FIG. 2A and results from the 4 hour incubation are reported in FIG. 2B. High SF values indicate increased fibroblast proliferation whereas low SF values indicate reduced fibroblast proliferation.

### Example 3: Formulation for Atopic Dermatitis

The following formulation may be used for atopic dermatitis of the face or body:

| Ingredient | w/w % |
|---|---|
| Water Phase | |
| Carboxyvinyl polymer | 0.3 |
| Propylene Glycol | 5.0 |
| Glycerine | 3.0 |
| Water | QS to 100% |
| Post Phasing additions: | |
| Water | QS to 100% |
| Amino Acids (AA) | Add single AAs or AA |
| | combinations and adjust water to |
| | equal 100% total formula |
| Methylparaben | 0.15 |
| Oil Phase | |
| White petrolatum | 3.0 |
| Propylparaben | 0.1 |
| Butylparaben | 0.05 |
| Cetyl palmitate | 3.0 |
| C12-C15 Alkyl Benzoate | 4.0 |
| Benzyl Alcohol | 0.5 |
| Glycerylmonostearate and | 5.0 |
| PEG100 stearate | |
| Stearyl alcohol | 0.5 |
| Ceatyl alcohol | 1.0 |

Water and oil phases are heated in separate vessels. The oil phase is added into water phase with stirring. The mixture is homogenized and stirred until uniform during cooling to room temperature. Post phasing additions are added to combined phases when cool. All ingredients are stirred and mixed for 15 minutes.

### Example 4: Formulation for Lip Treatment

The following formulation may be used as a lip treatment for, for example, treating oral herpes simplex:

| Ingredient | w/w % |
|---|---|
| Amino Acids (single or combinations) | 1-10 |
| Sucrose stearates | 11 |
| Sucrose Cocoate | 5 |
| Mineral Oil | 8 |
| Propylene Glycol | 5 |
| Benzyl Alcohol NF | 2.7 |
| Water | QS to 100% |

Water, and benzyl alcohol are mixed together and heated to 80° C. in one vessel, while the other ingredients are mixed and heated in a second vessel. The two phases are then combined with stirring to form a lotion/cream. The amino acids can be added after the lotion has cooled and has been stirred until uniform.

### Example 5: Formulation for Body Gel

The following formulation may be used as a body gel for, for example, treating atopic dermatitis:

| Ingredient | w/w % |
|---|---|
| Phase A | |
| Water, demin, | QS |
| Amino Acids (single or in combination) | 1-10 |
| Glycerin | 4.00 |
| PEG-8 | 8.00 |
| Aqua (and) Dimethicone (and) Dimethicone Crosspolymer (and) | 1.00 |
| Butylene Glycol (and) Hydrogenated Lecithin (and) | |
| Polyphosphorylcholine Glycol Acrylate | |
| Acrylates/beheneth-25 methacrylate copolymer | 3.3 |
| Phenoxyethanol, methylparaben, ethylparaben, propylene glycol | 1.25 |
| Phase B | |
| Dimethicone, cyclopentasiloxane, polysilicone-11 | 7.00 |
| Cetyl PEG/PPG-10/dimethicone | 1.00 |
| Octyldodecanol | 4.00 |
| Dimethicone | 3.00 |
| Phase C | |
| Water, demin. | 3.00 |
| Sodium hydroxide (25% solution) to neutralize to 6.5 pH | 0.45 |

Phase B is combined and mixed until uniform. Phase A is combined and mixed. Phase B is added to phase A while homogenizing. The mixture is homogenized until uniform. Mixing is continued and phase C is immediately added. The mixtures if mixed until uniform.

### Example 6: Formulation for Wound Healing

The following formulation may be used as a cream for, for example, healing wounds:

| Ingredients | w/w % |
|---|---|
| Phase A-1 | |
| Dimethicone (&) dimethicone/polyglycerin-3 crosspolymer | 4.00 |
| Methyl trimethicone (&) dimethicone/vinyl dimethicone | 1.00 |
| crosspolymer | |
| Polyglyceryl-3 polydimethylsiloxyethyl dimethicone | 1.20 |
| Phase A-2 | |
| Dimethicone | 3.00 |
| Methyl trimethicone | 7.00 |
| Phase B | |
| 1,3-Butylene glycol | 5.00 |
| Glycerin | 10.00 |
| Sodium citrate | 0.20 |
| Sodium chloride | 0.50 |
| Phenoxyethanol | 1.00 |
| Water | QS |
| Amino Acids (single or in combination) | 1-11 |
| Phase C | |
| TMF-1.5 | 5.00 |
| Methyl trimethicone (&) trimethylsiloxysilylcarbamoyl pullulan | 3.00 |

Phase A1 is combined and mixing is started. While mixing, Phase A2 is added to Phase A1 and mixed until smooth and uniform. In a separate vessel Phase B is combined and mixed well. To emulsify, Phase B is added to Phase A under low shear for 1-2 minutes and then speed is increased for 5 minutes. In a separate vessel Phase C is mixed until well blended then added to Phase A+B and mixed until well blended.

### Example 7: Keratinocyte Differentiation

Keratinocyte cultures may also be incubated with a range of doses of the single amino acids and/or the amino acid combinations 5AA (valine, threonine, tyrosine, serine, aspartic acid), 6AA (valine, threonine, tyrosine, serine, aspartic acid, tryptophan) or 8AA (valine, threonine, tyrosine, serine, aspartic acid, lysine, isoleucine, glycine) and assayed for the expression of cellular markers of keratinocyte differentiation. For primary human keratinocytes (PHK), such markers include keratin K1 and K10, involucrin, transglutaminase, zonular occludens 1, filaggrin, and, occludin. Such assays may be performed using, e.g., polymerase chain reaction amplification of RNA encoding each of these proteins or by western blot to visualize protein levels.

### Example 8: Disease Markers

Keratinocyte cultures may also be incubated with a range of doses of the single amino acids and/or the amino acid combinations 5AA (valine, threonine, tyrosine, serine, aspartic acid), 6AA (valine, threonine, tyrosine, serine, aspartic acid, tryptophan) or 8AA (valine, threonine, tyrosine, serine, aspartic acid, lysine, isoleucine, glycine) and assayed for the expression of various markers of disease to determine if the amino acid combinations, e.g., decrease expression of markers that are positively correlated with disease. Such markers of disease include, without limitation, nitric oxide synthase (NO52) and CCL27 (disease markers for psoriasis); and CCL27 (disease marker for eczema).

### Example 9: 3 Dimensional (3D) Model of Psoriasis

Psoriasis is a chronic skin disease that affects about 2% of the global population. An in vitro three-dimensional psoriatic-like tissue model may be purchased from MatTEk Corporation. Results generated this model system showed that the reconstructed psoriatic 3-D tissue model has phenotypic and architectural similarity to the in vivo counterpart in that the tissue model showed over expression of biomarkers associated with psoriasis including human beta defensin-2 (HBD-2), psoriasin, and CXCR2. Cytokine analysis of culture supernatants from the psoriatic-like tissue model showed increased release of IL-6 (7 fold), IL-8 (5.5 fold), and GRO-α (3.8 fold) when compared to normal reconstructed epidermal tissues. The psoriatic tissue model mimics the in vivo counterpart in terms of tissue morphology, tissue structure, gene expression (CXCR2, beta defensin, and psoriasin), and cytokine release (IL-6, IL-8, GRO-α) and thus, is considered to a model system in which to study the biology of psoriasis and for preclinical assessment of toxicity and proinflammatory effects of therapeutic candidates.

The examples and embodiments described herein are for illustrative purposes only and various modifications or changes in light thereof will be suggested to persons skilled in the art and are included within the spirit and purview of this application. In addition, any elements or limitations of any invention or embodiment thereof disclosed herein can be combined with any and/or all other elements or limitations (individually or in any combination) or any other invention or embodiment thereof disclosed herein, and all such combinations are contemplated with the scope of the invention without limitation thereto.

What is claimed is:

1. A method for treating a skin condition in a subject in need thereof, the method comprising administering to the subject a composition comprising, consisting essentially of, or consisting of a therapeutically effective amount of free amino acids of valine and serine; and
a therapeutically effective amount of one or more free amino acids comprising tyrosine, aspartic acid, threonine, tryptophan, lysine or isoleucine, or any combination thereof.

2. The method of claim 1, wherein the composition comprises, consists essentially of, or consists of a therapeutically effective amount of free amino acids of valine and serine; and
a therapeutically effective amount of two or more free amino acids comprising tyrosine, aspartic acid, threonine, tryptophan, lysine or isoleucine, or any combination thereof.

3. The method of claim 1, wherein the composition comprises, consists essentially of, or consists of a therapeutically effective amount of free amino acids of valine and serine; and
a therapeutically effective amount of three or more free amino acids comprising tyrosine, aspartic acid, threonine, tryptophan, lysine or isoleucine, or any combination thereof.

4. The method of claim 1, wherein the composition comprises, consists essentially of, or consists of a therapeutically effective amount of free amino acids of valine and serine; and
a therapeutically effective amount of four or more free amino acids comprising tyrosine, aspartic acid, threonine, tryptophan, lysine or isoleucine, or any combination thereof.

5. The method of claim 1, wherein the composition comprises, consists essentially of, or consists of a therapeutically effective amount of free amino acids of valine and serine; and
a therapeutically effective amount of five or more free amino acids comprising tyrosine, aspartic acid, threonine, tryptophan, lysine or isoleucine, or any combination thereof.

6. The method of claim 1, wherein the composition comprises, consists essentially of, or consists of a therapeutically effective amount of free amino acids of valine, serine, tyrosine, aspartic acid, threonine, tryptophan, lysine and isoleucine.

7. The method of claim 1, wherein the composition comprises, consists essentially of, or consists of a therapeutically effective amount of free amino acids of valine and serine; and
a therapeutically effective amount of one or more free amino acids comprising tyrosine, aspartic acid, or threonine, or any combination thereof.

8. The method of claim 1, wherein the composition comprises, consists essentially of, or consists of a therapeutically effective amount of free amino acids of valine and serine; and
a therapeutically effective amount of two or more free amino acids comprising tyrosine, aspartic acid, or threonine, or any combination thereof.

9. The method of claim 1, wherein the composition comprises, consists essentially of, or consists of a therapeutically effective amount of free amino acids of valine, serine, tyrosine, aspartic acid, and threonine.

10. The method of claim 1, wherein the composition comprises, consists essentially of, or consists of a therapeutically effective amount of free amino acids of valine, serine, tyrosine, aspartic acid, threonine, and tryptophan.

11. The method according to claim 1, wherein the composition comprises greater than zero mg/L asparagine to equal to or less than 0.1 mg/L asparagine, greater than zero mg/L alanine to equal to or less than 0.1 mg/L alanine, and/or greater than zero mg/L methionine to equal to or less than 0.1 mg/L methionine.

12. The method according to claim 1,
wherein the composition does not comprise asparagine, alanine, and/or methionine.

13. The method according to claim 1,
wherein the skin condition comprises atopic dermatitis, dermatitis, psoriasis, aging of skin, a condition related to the aging of skin, bed sores, pruritus, eczema, herpes simplex, fibrosis, a wound, or a burn.

14. The method according to claim 1,
wherein the skin condition is a cosmetic skin condition.

15-20. (canceled)

21. The method according to claim 1, wherein each of the free amino acids is at a concentration of from about 0.1 to 2.0 grams/liter.

22. The method according to claim 1, wherein the composition does not include, or comprises only negligible amounts of electrolytes.

23. The method according to claim 1, wherein the subject is a human.

24. The method according to claim 1, wherein the composition is administered to the subject via transdermal, subcutaneous, or topical administration.

25. The method according to claim 1, wherein the composition is administered on a daily dosing schedule.

26. The method according to claim 1, wherein the composition is sterile.
